(19) United States
(12) Patent Application Publication
(10) Pub. No.: US 2024/0170101 A1
(43) Pub. Date: May 23, 2024

(54) SPECTRAL CORRELATION ANALYSIS OF LAYERED EVOLUTIONARY SIGNALS

(71) Applicants: The University of Chicago, Chicago, IL (US); Washington University, St. Louis, MO (US)

(72) Inventors: Arjun S. RAMAN, Chicago, IL (US); Mark A. ZAYDMAN, St. Louis, MO (US)

(73) Assignees: The University of Chicago, Chicago, IL (US); Washington University, St. Louis, MO (US)

(21) Appl. No.: 18/550,797

(22) PCT Filed: Mar. 16, 2022
(86) PCT No.: PCT/US22/71184

§ 371 Date: Sep. 15, 2023

Related U.S. Application Data
(60) Provisional application No. 63/161,786, filed on Mar. 16, 2021.
(60) Provisional application No. 63/165,006, filed on Mar. 23, 2021.

(51) Int. Cl. G16B 40/30; G16B 20/20; G16B 30/10

(52) U.S. Cl. CPC G16B 40/30; G16B 20/20; G16B 30/10

(57) ABSTRACT

Aspects of the disclosure include analysis methods and systems useful in, for example, determining protein-protein interaction, characterizing emergent biological function, and discovery of novel protein function. Aspects include generating a spectral matrix representing variation of genetic elements based at least in part on singular value decomposition (SVD) of a first matrix comprising entries representing a number of times each of a genetic element appears in each of a detectable proteome. Biological information contained in one or more cells of the spectral matrix may be quantified to obtain statistical interactions between the genetic elements. The obtained statistical interactions may be correlated with benchmarked biological interactions. Accordingly, a protein-protein interaction may be classified using correlations between the statistical interactions and the benchmarked biological interactions. Emergent function may be characterized and protein function may be predicted by further analysis of the spectral matrix.

### CROSS-REFERENCE TO RELATED APPLICATIONS

This application claims the benefit of priority of U.S. Provisional Patent Application No. 63/161,786 filed Mar. 16, 2021, and U.S. Provisional Patent Application No. 63/165,006 filed Mar. 23, 2021, which are hereby incorporated by reference in their entirety.

### BACKGROUND OF THE INVENTION

### I. Field of the Invention

Aspects of the present disclosure relate to at least the fields of computational biology and bioinformatics. More particularly, aspects of the disclosure relate to analysis and characterization of emergent function based on individual components of biological systems such as protein-protein interactions.

### II. Background

A fundamental problem in biology is to understand how proteins interact to create a complex phenotype (Barabasi and Oltvai, 2004; Chuang et al., 2010; Hartwell et al., 1999; Costanzo et al., 2016). Biochemical and genetic studies have illustrated that complex behaviors emerge from a hierarchy of protein interactions: proteins interact to form complexes, complexes interact to form pathways, and pathways interact to create phenotypes (Papin et al., 2004; Ravasz 2009; Nurse 2008). Current strategies for identifying protein interactions span both experiment and computation. Experimental methods identify protein-protein interactions (PPIs) across different model systems and are continuing to evolve to be more high-throughput and comprehensive (Rajagopala et al., 2014; Schoenrock et al., 2017; Hauser et al., 2014; Koo et al., 2017; Luck et al., 2020). Computational methods leverage covariation between orthologous genes (orthologs) to predict PPIs (Eisen, 1998; Pellegrini et al, 1999; Enrich et al., 1999; Valencia and Pazos, 2002). Advances in computation as well as the breadth of available data have fueled continued innovation such as using statistical physical methods to infer PPIs from amino-acid coevolution (Croce et al., 2019; Cong et al., 2019; Green et al., 2021).

Pairwise PPIs derived from experimental and computational approaches are used to infer higher-order interaction networks (Szklarczyk et al., 2018). However, recent experimental work has shown that protein interaction networks defined only by binary interactions are incomplete, suggesting that important biological information lies in higher-order protein interactions (Kuzmin et al., 2018). Therefore, creating a model of genotype to phenotype relationships requires the ability to identify different ‘scales’ of interactions, from pairwise to higher-order, and relating these scales to describe the integration of pairwise interactions into higher-order interactions.

Recognized is a need for methods and systems for investigating, analyzing, and characterizing higher-order biological interactions.

### SUMMARY

In view of the foregoing, aspects of the present disclosure provide methods, systems, and computer-readable media that analyze higher-order biological interactions and are useful in, for example, identification of complex emergent phenotypes and prediction of individual protein function based on Singular Value Decomposition analysis of covariation between protein orthologs.

A particular implementation provides a method of determining interaction between proteins. The method includes receiving, at a first module, inputs corresponding to detectable proteomes of organisms. The inputs may be derived from sequencing genomes of the organisms. The inputs may be derived from proteomic analysis (e.g., mass spectrometry analysis) of the organisms. The method also includes generating, at the first module, a first matrix comprising a plurality of rows and a plurality of columns. In some aspects, each of the plurality of rows corresponds to a detectable proteome of an organism. In some aspects, each of the plurality of columns corresponds to a genetic element. An entry in the first matrix may represent a number of times each genetic element appears in each detectable proteome. “Genetic element” describes any component(s) or element(s) of a gene, transcript, or protein useful in identification and comparison with other genes, transcripts, and/or proteins. The method includes performing, at a second module, singular value decomposition (SVD) on the first matrix to generate a second matrix. In some aspects, the second matrix is a spectral matrix representing a variation for each genetic element. The method includes quantifying, at a third module, biological information contained in one or more cells of the spectral matrix by calculating pair-wise correlations for the genetic elements to obtain statistical interactions between the genetic elements at different scales of variation. “Biological information” describes any information related to the biology of organism(s), including phylogenetic relationships, interactions between proteins in pathways, physical interactions within protein complexes, etc. The method also includes correlating, at a fourth module, the obtained statistical interactions with benchmarked biological interactions. “Benchmarked biological interactions” describes biological information obtained based on known, existing biological relationships. The method includes classifying, at a fifth module, a protein-protein interaction using the correlations between statistical interactions and the benchmarked biological interactions. The method further includes displaying information indicating the classification to a user.

In some aspects, the organisms comprise one or more of prokaryotic organisms, eukaryotic organisms, and archaeal organisms. In some aspects, the organisms are prokaryotic organisms. In some aspects, the organisms are eukaryotic organisms. In some aspects, the organisms are mammalian organisms. Example genetic elements include, but are not limited to, orthologous gene groups, conserved proteins domains, conserved sequences, and conserved protein function. In some aspects, the genetic element is or includes an orthologous gene group. In some aspects, the genetic element is or includes a conserved protein domain.

In some aspects, the SVD on the first matrix further produces a scaling for the spectral matrix showing that fractional variance of at least some components is linearly related to a component number, wherein these components are used in the quantifying step. In some aspects, the pair-wise correlations for the genetic elements are calculated within all five-component windows of the one or more cells of the spectral matrix. In some aspects, the benchmarked biological interactions include phylogenetic relationships, indirect protein interactions in cellular pathways, direct protein interaction, or a mixture of indirect and direct interactions. In some aspects, the correlating step is conducted by quantifying mutual information shared between the statistical interactions and benchmarked biological interactions.

In some aspects, the classifying is conducted using one or more trained Random Forest models. The one or more trained Random Forest models may be trained via steps of: dividing the spectral matrix obtained from the SVD into two or more mutual information windows, each of which is enriched for information representing at least one biological interaction; and computing spectral correlations for each genetic element pair over each mutual information window. In some aspects, the one or more trained Random Forest models are trained using orthologous gene groups. In some aspects, the one or more trained Random Forest models are trained using conserved protein domains. In some aspects, the Random Forest models are trained using protein interaction information of an organism. For example, where the methods comprise analysis of protein-protein interaction in E. coli, the Random Forest models may be trained using protein interaction information of E. coli K12.

A further implementation provides a method for characterizing an emergent biological function. “Emergent biological function” describes any function related to the biology of organism(s) based on integration of individual components (e.g., individual proteins, individual protein-protein interactions, etc.) into a collective unit of function. The method includes developing, at a sixth module, a null model for protein correlations over a predetermined window of a spectral matrix using a method for determining protein interactions as disclosed herein. The method further comprises identifying, at a seventh module, proteins that are statistically significantly correlated with a protein for the emergent biological function. The method comprises determining, at an eighth module, a spectral depth of correlation for all pairs of identified proteins by identifying a first window of the spectral matrix where correlation value for all pairs of the identified proteins is below the threshold of statistical significance. The method further comprises identifying, at a ninth module, networks of proteins sharing a selected spectral depth of correlation to obtain statistical modules. In some aspects, the sets of proteins within each statistical module are connected to each other with a denser connection than to proteins outside the statistical module. The method comprises assigning, at a tenth module, a function to each of the statistical module using gene set enrichment analysis. The method further comprises obtaining, at an eleventh module, hierarchical interactions in a pathway for the emergent biological function by comparing the assigned functions at various spectral depths. The method further includes displaying information indicating the hierarchical interactions for the emergent biological function to a user.

In some aspects, the predetermined window overlaps with an indirect interaction mutual information window for orthologous gene group variation. In some aspects, the predetermined window contains pathway-level interaction information. The method further comprises, in some aspects, at a twelfth module, characterizing a protein in the statistical model as having the function of the statistical model based on the gene set enrichment analysis. The method further includes, in some aspects, displaying information indicating the function of the protein to the user.

Further disclosed are non-transitory computer-readable storage medium storing instructions that, when executed by one or more processors, cause the one or more processors to perform operation for performing one or more methods disclosed herein.

Throughout this application, the term “about” is used to indicate that a value includes the inherent variation of error for the measurement or quantitation method.

The use of the word “a” or “an” when used in conjunction with the term “comprising” may mean “one,” but it is also consistent with the meaning of “one or more,” “at least one,” and “one or more than one.”

The phrase “and/or” means “and” or “or”. To illustrate, A, B, and/or C includes: A alone, B alone, C alone, a combination of A and B, a combination of A and C, a combination of B and C, or a combination of A, B, and C. In other words, “and/or” operates as an inclusive or.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. Compositions and methods “consisting essentially of” any of the ingredients or steps disclosed limits the scope of the claim to the specified materials or steps which do not materially affect the basic and novel characteristic of the claimed invention.

It is contemplated that any embodiment discussed in this specification can be implemented with respect to any method or composition of the invention, and vice versa. Furthermore, compositions of the invention can be used to achieve methods of the invention.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1C. The SVD spectrum of OGG variation organized covariation according to biological scale. (FIG. 1A) The data matrix, DOGG, contained the number of annotations of each of 10,177 orthologous gene groups (OGGs) within each of 7,047 UniProt bacterial reference proteomes. (FIG. 1B) Benchmarks were assembled to represent prior knowledge of phylogenetic relationships (Phylogeny), indirect PPIs (Indirect PPI), and direct PPIs (Direct PPI). For each benchmark, black circles indicate the types of information represented. (FIG. 1C) Cumulative distribution functions for the mutual information (MI cdfs) shared between the SVD components of DOGG and the benchmarks in FIG. 1B. Shaded regions indicate +2 standard deviations surrounding the mean value for bootstraps of the benchmark. Components of covariation explain progressively less of the overall data variance with increasing spectral position.

FIG. 2. Phylogenetic tree of the 7,047 bacterial species represented in the UniProt reference proteomes database (release 02/2020) that served as the substrate for DOGG (shown in FIG. 1A). The tree was generated using PhyloT based on the NCBI taxonomy database and visualized using iTOL (Letunic et al., 2006).

FIGS. 3A-3D. Using SVD to spectrally decompose OGG covariation in bacteria. (FIG. 3A) The raw OGG content matrix, DOGG. (FIG. 3B) The z-scored OGG content matrix, ZOGG produced by subtracting the column mean from each element in DOGG and then dividing by the column standard deviation. (FIG. 3C) Application of SVD to ZOGG produced three matrices: UOGG, ΣOGG, and VOGG. UOGG contains the left singular vectors (LSVs) describing the projections of each proteome onto the SVD components. ZOGG is a diagonal matrix with decreasing diagonal elements containing the singular values (inset expands top 20 singular values). The magnitude of each singular value is related to the fraction of the overall data variance explained by the corresponding SVD component. VOGG contains the right singular vectors (RSVs) describing the projections of each OGG onto the SVD components. (FIG. 3D) Percent variance explained per component versus component number. Dotted line represents the fit to a power-law distribution with the indicated exponent (γ).

FIG. 4. Projection of proteomes onto SVD1, SVD2, SVD3, and SVD4 of DOGG colored by phylum. Proteome projections onto the SVD components are derived from UOGG, the matrix of left singular vectors (LSVs) defined by applying SVD to DOGG. Each marker represents a single proteome and is colored according by Phylum as indicated. The percent variance explained per SVD component is indicated in parentheses.

FIGS. 5A-5G. The process of computing spectral correlations between two proteins within a specific region of the SVD spectrum of DOGG. (FIGS. 5A-5B) The OGG structures of E. coli K12 ArgA (FIG. 5A) and ArgH (FIG. 5B). (FIGS. 5C-5D) The projections (T) of the OGGs encoded in ArgA (COG0548 and COG1246) (FIG. 5C) and ArgH (COG0165) (FIG. 5D) onto SVD1 to SVD200 of the SVD spectrum of DOGG. (FIGS. 5E-5F) The approximated projections (Ω) of ArgA (FIG. 5E) and ArgH (FIG. 5F) derived by averaging the projections for the OGGs encoded within each protein. (FIG. 5G) Overlay of the approximated protein projections of ArgA and ArgH. These two protein project similarly across SVDI to SVD200 resulting in a positive spectral correlation value (Pearson correlation value shown).

FIG. 6. Estimating the MI shared between spectral correlations and a benchmark. (Top) Hypothetical projection matrix Mdata consists of projections of six variables onto six SVD components. If the variables correspond to the rows or columns of D, the initial data matrix, then the matrix Mdata corresponds to the complete U or V matrices produced by application of SVD to D, respectively. Mdata is windowed to components 3 to 5 to produce Mdata3:5 by discarding all columns outside of this range. Next Pearson correlations are computed between all pairs of rows in Mdata3:5 to produce the windowed spectral correlation matrix Cdata3:5. The MI shared between Cdata3:5 and a benchmark reflects the degree to which the distribution of spectral correlation values in Cdata3:5 differs for variable pairs that interact within the benchmark (benchmark positive) and variable pairs that do not interact in the benchmark (benchmark negative). (Bottom) To estimate the MI produced by spurious correlations, Mdata is subjected to random row permutation to generate Mshuffled. This process maintains the distribution of projection values for each variable but erases biologically meaningful spectral correlations leaving only the spurious correlations. Mshuffled is subjected to the identical windowing, row correlation computation, and MI calculations as described above for Mdata. The biologically meaningful MI is estimated to be the difference between the MI estimate for Cdata3:5 and 3:5 Cshuffled.

FIGS. 7A-7B. Quantifying the biological information contained within different regions of the SVD spectrum of DOGG. (FIG. 7A) MI density contained within a 5-component window versus spectral position for all windows between SVD1 and SVD3000 in the SVD spectrum of DOGG. Colored and gray lines represent the MI values for the data matrix or the model of spurious spectral correlations, respectively. Lines and shaded contours represent the mean ±2 standard deviations for the bootstraps of each benchmark. (FIG. 7B) MI density contained within SVD2995-SVD3000 of the SVD spectrum of DOGG. Each dot represents the MI value for a single bootstrap of the indicated benchmark. Colored dots represent the MI for the data matrix and gray dots represent the MI for the model of spurious correlations. P-values produced by a paired Student's t-test are indicated.

FIGS. 8A-8D. MI windowed spectral correlations (MIWSCs) enable accurate classification of protein pairs as either not-interacting, indirect PPI, or direct PPI. (FIG. 8A) Fractional MI density regarding each benchmark contained within spectral correlations computed across SVD1-33, SVD34-223, or SVD234-3000 of DOGG. (FIG. 8B) F-scores for predicting interaction classes for pairs in an independent validation set of E. coli K12 proteins using RF models trained on either MIWSCs, quantitative features of published datasets derived from experimental methods (gene epistasis [epistasis], yeast-two-hybrid [Y2H], affinity purification mass spectrometry [APMS1, APMS2]), quantitative features of published datasets derived from computational methods (gene cooccurrence [GC], gene fusion [GF], gene neighborhood [GN]), or quantitative features of established computational methods derived de novo from DOGG (binary mutual information [b-MI], covariation [Cov], Principle Components Analysis including components 1-k [PCA]). The violin plots describe the distribution of F-scores for models trained and validated on 50 random partitions of the gold-standard dataset. Numbering indicates the rank of the median F-score for models trained on each feature. (FIG. 8C) Precision (left) and recall (right) for direct PPI predictions in 12 phylogenetically diverse organisms using RF models trained on the MIWSCs of E. coli K12 proteins benchmarked against the experimentally supported PPIs in the STRING database. Comparisons are made to a set of 10,000 randomly selected pairs and to the ‘high confidence’ predictions in the STRING database subchannels for the methods of gene cooccurrence (GC), gene neighborhood (GN), and gene fusion (GF). Vertical dashed line indicates the median value for RF models trained on MIWSCs. ** in legend indicates an organism that was not part of the input dataset DOGG. (FIG. 8D) Percent of predicted direct PPIs in M. tuberculosis H37Rv supported by an absent (0), low (0 to 0.4), or high (>0.4) composite score (left) or an absent (0) or present (>0) experimental subchannel score (right) in the STRING database. Comparisons were made between the methods of random selection (Random), amino acid coevolution ('Cong et al.', Cong et al., 2019), or RF models trained on MIWSC features of E. coli K12 proteins (MIWSC). Numbers of predicted interactions in each bin are indicated.

FIG. 9. Multi-level classification task where RF models were trained and validated for predicting indirect and direct PPIs in E. coli K12 using spectral correlations features, existing computational features, or existing experimental features. A gold-standard dataset of well-characterized E. coli K12 protein pairs was assembled and partitioned into training and validation datasets. The labeled examples from the training set were used to train RF models to classify protein interactions using different features as indicated. The performance of the various RF models was benchmarked and compared by computing F-scores for classifying PPIs in the validation dataset, out-of-bag examples in the training dataset, and PPIs in four additional comprehensive benchmarks. This process of partitioning the gold-standard dataset, training, and validation was repeated 50 times to evaluate the reproducibility of RF model performance.

FIGS. 10A-10B. Extracting MIWSC features for the training and validation of RF models to classify E. coli K12 protein pairs as not-interacting (‘not-interacting’), indirect PPI (‘indirect’), or direct PPI (‘direct’). (FIG. 10A) A gold standard dataset of well characterized E. coli K12 protein pairs was assembled and randomly partitioned in to training (60%) and validation (40%) datasets. An MIWSC feature was extracted for each protein pair in the training and validation partitions of the gold standard dataset. The MIWSC feature consists of a set of three spectral correlations computed across SVD1-33, SVD34-223, and SVD234-3000. Each pixel of the heat map is the spectral correlation for the protein pair indicated in the row across the SVD components indicated in the column. (FIG. 10B) Using only the MIWSC features, RF models were trained using the labeled examples in the training dataset and then challenged to predict the unlabeled examples in the validation dataset.

FIGS. 11A-11B. F-scores for predicting interaction classes for out-of-bag examples in the training datasets (FIG. 11A) and four additional comprehensive benchmarks (FIG. 11B). The violin plots describe the distribution of F-scores for models trained and validated on 50 random partitions of the gold-standard dataset. Numbering indicates the rank of the median F-score for models trained on each feature.

FIGS. 12A-12C. The SVD spectrum of protein domain variation organized covariation according to biological scale. (FIG. 12A) The domain content matrix, Ddomain, contained the number of annotations of each of 7,245 conserved protein domains within each of the 7,047 UniProt bacterial reference proteomes. (FIG. 12B) Percent variance explained per component versus component number for the SVD spectra of DOGG (black) and Ddomain (gray). Dotted lines represent a power-law distribution with the indicated exponent (γ). (FIG. 12C) Cumulative distribution functions for the mutual information (MI cdfs) shared between the SVD components of Ddomain and the indicated benchmarks. Shaded regions indicate ±2 standard deviations surrounding the mean value for bootstraps of each benchmark (STAR Methods).

FIGS. 13A-13B. Quantifying the biological information contained within different regions of the SVD spectrum of Ddomain. (FIG. 13A) MI density contained within a 5-component window versus spectral position for all windows between SVD1 and SVD3000 in the SVD spectrum of Ddomain. Colored and gray lines represent the MI values for the data matrix or the model of spurious spectral correlations, respectively. Lines and shaded contours represent the mean ±2 standard deviations for the bootstraps of each benchmark. (FIG. 13B) MI density contained within SVD2995-SVD3000 of the SVD spectrum of Ddomain. Each dot represents the MI value for a single bootstrap of the indicated benchmark. Colored dots represent the MI for the data matrix and gray dots represent the MI for the model of spurious correlations. P-values produced by a paired Student's t-test are indicated.

FIGS. 14A-14D. Domain-based MI windowed spectral correlations (MIWSCsdomain) enable accurate classification of protein pairs as either not-interacting, indirect PPI, or direct PPI. (FIG. 14A) Fractional MI density regarding each benchmark contained within spectral correlations computed across SVD1-22, SVD23-358, or SVD359-3000 of Ddomain. (FIGS. 14B-14D) F-scores for classifying protein pairs in the validation dataset (FIG. 14B), out-of-bag examples from the training dataset (FIG. 14C), or additional examples in four comprehensive benchmarks (FIG. 14D). The violin plots describe the distribution of F-scores for models trained and validated on 50 random partitions of the gold-standard dataset. Numbering indicates the rank of the median F-score for models trained on each feature.

FIGS. 15A-15B. A hierarchical model of E. coli K12 motility derived by serially thresholding ‘spectral depth’ resembles that described in the KEGG database. (FIG. 15A) The model contained 75 proteins that were identified as significantly correlated with FliC across SVD34 to SVD134. Statistical interaction networks were defined by thresholding spectral depth at 50 (top panel), 300 (middle panel), and 1000 (bottom panel). Nodes (yellow circles) represent proteins and edges (red lines) represent statistical interactions. Shaded contours identify discrete subnetworks and are labeled with their assigned function based on interpretation of gene-set enrichment analysis (GSEA) and literature review. The most significantly enriched ontological term produced by GSEA and the associated p-value is shown in parentheses for each subnetwork. (FIG. 15B) Comparison of the statistically-derived model (left) to the KEGG model (BR:eco02035, right) of E. coli K12 motility. Venn diagrams represent the overlap between the sets of proteins in the indicated subnetwork of FIG. 15A (left) and the indicated KEGG category (right).

FIGS. 16A-16E. Developing a null model of random protein-protein spectral correlations within the SVD spectrum of DOGG. (FIG. 16A) Cumulative distribution functions (cdfs) for spectral correlations between all proteins in E. coli K12 across windows of different widths (legend) centered on component 1001. (FIG. 16B) Variance of the distributions in FIG. 16A plotted versus window width. Vertical line indicates a window width of 100 components. (FIG. 16C) cdfs for spectral correlations between all proteins in E. coli K12 across the indicated 100-component spectral windows (legend). (FIG. 16D) cdfs for spectral correlations for all proteins in proteomes from diverse organisms (legend) across the 100-component window centered on component 84. (FIG. 16E) p-value versus correlation threshold for spectral correlations between proteins in E. coli K12 across SVD34 to SVD134. The chosen correlation threshold (0.29) and associated p-value (0.018) are indicated.

FIGS. 17A-17D. Process for constructing the FliC interaction networks appearing in FIG. 15 using thresholded spectral depth. (FIG. 17A) Proteins that shared significant spectral correlations with FliC across SVD34 to SVD134. (FIG. 17B) Pairwise spectral correlations between indicated protein pairs (legend) as a function of spectral position. Horizontal dashed line represents the threshold of significant spectral correlation described in FIG. 16E. Dashed vertical lines represent the ‘spectral depth’—the spectral position at which spectral correlation first falls below the significance threshold. (FIG. 17C) Hierarchically clustered spectral depth matrix for all pairs of proteins in FIG. 17A. (FIG. 17D) Set of adjacency matrices derived from thresholding the spectral depth matrix in FIG. 17C. Red and white pixels indicate proteins that do or do not share a spectral depth exceeding the indicated thresholds respectively.

FIGS. 18A-18C. A statistically-derived hierarchical model of motility in E. coli K12 using MotB as a query protein. (FIG. 18A) 60 proteins in E. coli K12 shared significant spectral correlations with MotB across SVD34 to SVD134. Statistical interaction networks were defined by thresholding spectral depth at 50 (top panel), 300 (middle panel), and 1000 (bottom panel). Nodes (yellow circles) represent proteins and edges (red lines) represent statistical interactions. Shaded contours identify discrete subnetworks and are labeled with their assigned function based on interpretation of gene-set enrichment analysis (GSEA) and literature review. The most significantly enriched ontological term produced by GSEA and the associated p-value is shown in parentheses for each subnetwork. (FIG. 18B) Comparison of the statistically-derived model using MotB (left) to the KEGG model (BR:eco02035, right) of E. coli K12 motility. Venn diagrams represent the overlap between the sets of proteins in the indicated subnetwork of panel A (left) and the indicated KEGG category (right). (FIG. 18C) Pie graph of the number of proteins in the statistical model that are represented in the KEGG hierarchy (‘KEGG’), missing from the KEGG hierarchy but supported by experimental evidence in the literature (‘Literature’), or absent from the KEGG hierarchy and the literature (‘Novel’).

FIGS. 19A-19C. A statistically-derived hierarchical model of motility in B. subtilis 168 using Hag as a query protein. (FIG. 19A) 74 proteins in B. subtilis 168 shared significant spectral correlations with Hag over SVD34 to SVD134. Statistical interaction networks were defined by thresholding spectral depth at 50 (top panel), 300 (middle panel), and 1000 (bottom panel). Nodes (yellow circles) represent proteins and edges (red lines) represent statistical interactions. Shaded contours identify discrete subnetworks and are labeled with their assigned function based on interpretation of gene-set enrichment analysis (GSEA) and literature review. The most significantly enriched ontological term produced by GSEA and the associated p-value is shown in parentheses for each subnetwork. (FIG. 19B) Comparison of the statistically-derived model using Hag (left) to the KEGG model (BR:bsu02035, right) of B. subtilis 168 motility. Venn diagrams represent the overlap between the sets of proteins in the indicated subnetwork of panel A (left) and the indicated KEGG category (right). (FIG. 19C) Pie graph of the number of proteins in the statistical model that are represented in the KEGG hierarchy (‘KEGG’), missing from the KEGG hierarchy but supported by experimental evidence in the literature (‘Literature’), or absent from the KEGG hierarchy and the literature (‘Novel’).

FIGS. 20A-C. A statistically-derived hierarchical model of amino acid metabolism in E. coli K12 using HisG as a query protein. 129 proteins in E. coli K12 shared significant spectral correlations with HisG across components SVD34 to SVD134. Statistical interaction networks were defined by thresholding spectral depth at 50, 150, 300, and 1000. Nodes (yellow circles) represent proteins and edges (red lines) represent statistical interactions. Shaded contours identify discrete subnetworks and are labeled with their assigned function based on interpretation of gene-set enrichment analysis (GSEA) and literature review. The most significantly enriched ontological term produced by GSEA and the associated p-value is shown in parentheses for each subnetwork.

FIGS. 21A-21B. Statistically-derived hierarchical models of bacterial motility derived from serially thresholding spectral depth for correlations in Ddomain. (FIG. 21A) A model of motility in B. subtilis 168 using Hag as a query. (FIG. 21B) A model of motility in E. coli K12 using FliC as a query. Statistical interaction networks were defined by thresholding spectral depth at 50, 300, and 1000. Nodes (yellow circles) represent proteins and edges (red lines) represent statistical interactions. Shaded contours identify discrete subnetworks and are labeled with their assigned function based on interpretation of gene-set enrichment analysis (GSEA) and literature review. The most significantly enriched ontological term produced by GSEA and the associated p-value is shown in parentheses for each subnetwork.

FIGS. 22A-22C. Prediction and experimental validation of a novel effector of twitch-based motility in P. aeruginosa (PAO1). (FIG. 22A) Statistical network derived by applying a spectral depth threshold of 300 to the set of 141 protein in P. aeruginosa that were significantly correlated with PilA across SVD34 to SVD134. Nodes (yellow circles) represent proteins and edges (red lines) represent statistical interactions. Shaded contours identify statistical subnetworks and are labeled with their assigned function based on interpretation of gene-set enrichment analysis (GSEA) and literature review. The most significantly enriched ontological term produced by GSEA and the associated p-value is shown in parenthesis for each subnetwork. (FIG. 22B) The pilus motility subnetwork from panel A. Nodes representing PilA and Q915G6 are colored blue and green respectively. (FIG. 22C) Time-course of pilus-based motility for parent (WT), two transposon mutants of Q915G6 (Tn(1) Q915G6, Tn(2) Q915G6), and transposon mutants complemented with Q915G6 (Tn(1) Q915G6 +Q915G6, Tn(2) Q915G6 +Q915G6). Inset shows results of flagellar motility for the parent strain (WT), and the two transposon mutants of Q915G6 (Tn(1), Tn(2)) 24 hours post-inoculation. Representative images of the crystal-violet stained plates are shown.

FIGS. 23A-23B. Statistically-derived hierarchical model of directed-motility in P. aeruginosa using PilA as a query. 140 proteins in P. aeruginosa shared significant spectral correlations with PilA across SVD34 to SVD134. (FIG. 23A) Statistical interaction network defined by thresholding spectral depth at 50. The inset illustrates significantly enriched terms resulting from gene-set enrichment analysis (GSEA) of the entire network. (FIG. 23B) Statistical interaction network defined by thresholding spectral depth at 300. Nodes (yellow circles) represent proteins and edges (red lines) represent statistical interactions. Shaded contours identify discrete subnetworks and are labeled in FIG. 23B with their assigned function based on interpretation of GSEA and literature review. The most significantly enriched ontological term produced by GSEA and the associated p-value is shown in parentheses for each subnetwork.

FIG. 24 is a flow diagram illustrating an example of a method for determining interaction between proteins.

FIG. 25 is a block diagram illustrating an example of a system configured for determining interaction between proteins.

### DETAILED DESCRIPTION

Aspects of the present disclosure are directed to analysis of higher-order biological interactions. Certain aspects provide systems, methods, and computer-readable media directed to determining interaction between proteins, characterizing emergent biological function, and/or characterizing or identifying protein or gene function based on analysis of inputs corresponding to detectable proteomes of organisms. For example, singular value decomposition may be performed to generate a spectral matrix representing variation for each of a plurality of genetic elements (e.g., orthologous gene groups, conserved protein domains, etc.) and used to quantify biological information to obtain statistical interactions between the genetic elements at different scales of variation. By correlating the statistical interactions with benchmarked biological interactions, protein-protein interaction may be classified and information indicating the classification displayed.

### I. Detecting a Genetic Signature

Particular embodiments concern methods of detecting a genetic signature in an individual or organism, in some cases followed by analysis of the genetic signature. In some embodiments, the method for detecting the genetic signature may include selective oligonucleotide probes, arrays, allele-specific hybridization, molecular beacons, restriction fragment length polymorphism analysis, enzymatic chain reaction, flap endonuclease analysis, primer extension, 5′-nuclease analysis, oligonucleotide ligation assay, single strand conformation polymorphism analysis, temperature gradient gel electrophoresis, denaturing high performance liquid chromatography, high-resolution melting, DNA mismatch binding protein analysis, surveyor nuclease assay, sequencing, or a combination thereof, for example. The method for detecting the genetic signature may include fluorescent in situ hybridization, comparative genomic hybridization, arrays, polymerase chain reaction, sequencing, or a combination thereof, for example. The detection of the genetic signature may involve using a particular method to detect one feature of the genetic signature and additionally use the same method or a different method to detect a different feature of the genetic signature. Multiple different methods independently or in combination may be used to detect the same feature or a plurality of features.

### Single Nucleotide Polymorphism (SNP) Detection

Particular embodiments of the disclosure concern methods of detecting a SNP. One may employ any of the known general methods for detecting SNPs for detecting the particular SNP in this disclosure, for example. Such methods include, but are not limited to, selective oligonucleotide probes, arrays, allele-specific hybridization, molecular beacons, restriction fragment length polymorphism analysis, enzymatic chain reaction, flap endonuclease analysis, primer extension, 5′-nuclease analysis, oligonucleotide ligation assay, single strand conformation polymorphism analysis, temperature gradient gel electrophoresis, denaturing high performance liquid chromatography, high-resolution melting, DNA mismatch binding protein analysis, surveyor nuclease assay, sequencing, or a combination thereof.

In some embodiments of the disclosure, the method used to detect the SNP comprises sequencing nucleic acid material and/or using selective oligonucleotide probes. Sequencing the nucleic acid material may involve obtaining the nucleic acid material in the form of genomic DNA, complementary DNA that is reverse transcribed from RNA, or RNA, for example. Any standard sequencing technique may be employed, including Sanger sequencing, chain extension sequencing, Maxam-Gilbert sequencing, shotgun sequencing, bridge PCR sequencing, high-throughput methods for sequencing, next generation sequencing, RNA sequencing, or a combination thereof. After sequencing the nucleic acid, one may utilize any data processing software or technique to determine which particular nucleotide is present at the particular SNP.

In some embodiments, the nucleotide at the particular SNP is detected by selective oligonucleotide probes. The probes may be used on nucleic acid material, including genomic DNA, complementary DNA that is reverse transcribed from RNA, or RNA, for example. Selective oligonucleotide probes preferentially bind to a complementary strand based on the particular nucleotide present at the SNP. For example, one selective oligonucleotide probe binds to a complementary strand that has an A nucleotide at the SNP on the coding strand but not a G nucleotide at the SNP on the coding strand, while a different selective oligonucleotide probe binds to a complementary strand that has a G nucleotide at the SNP on the coding strand but not an A nucleotide at the SNP on the coding strand. Similar methods could be used to design a probe that selectively binds to the coding strand that has a C or a T nucleotide, but not both, at the SNP. Thus, any method to determine binding of one selective oligonucleotide probe over another selective oligonucleotide probe could be used to determine the nucleotide present at the SNP.

One method for detecting SNPs using oligonucleotide probes comprises the steps of analyzing the quality and measuring quantity of the nucleic acid material by a spectrophotometer and/or a gel electrophoresis assay; processing the nucleic acid material into a reaction mixture with at least one selective oligonucleotide probe, PCR primers, and a mixture with components needed to perform a quantitative PCR (qPCR), which could comprise a polymerase, deoxynucleotides, and a suitable buffer for the reaction; and cycling the processed reaction mixture while monitoring the reaction. In one embodiment of the method, the polymerase used for the qPCR will encounter the selective oligonucleotide probe binding to the strand being amplified and, using endonuclease activity, degrade the selective oligonucleotide probe. The detection of the degraded probe determines if the probe was binding to the amplified strand.

Another method for determining binding of the selective oligonucleotide probe to a particular nucleotide comprises using the selective oligonucleotide probe as a PCR primer, wherein the selective oligonucleotide probe binds preferentially to a particular nucleotide at the SNP position. In some embodiments, the probe is generally designed so the 3′ end of the probe pairs with the SNP. Thus, if the probe has the correct complementary base to pair with the particular nucleotide at the SNP, the probe will be extended during the amplification step of the PCR. For example, if there is a T nucleotide at the 3′ position of the probe and there is an A nucleotide at the SNP position, the probe will bind to the SNP and be extended during the amplification step of the PCR. However, if the same probe is used (with a T at the 3′ end) and there is a G nucleotide at the SNP position, the probe will not fully bind and will not be extended during the amplification step of the PCR.

In some embodiments, the SNP position is not at the terminal end of the PCR primer, but rather located within the PCR primer. The PCR primer should be of sufficient length and homology in that the PCR primer can selectively bind to one variant, for example the SNP having an A nucleotide, but not bind to another variant, for example the SNP having a G nucleotide. The PCR primer may also be designed to selectively bind particularly to the SNP having a G nucleotide but not bind to a variant with an A, C, or T nucleotide. Similarly, PCR primers could be designed to bind to the SNP having a C or a T nucleotide, but not both, which then does not bind to a variant with a G, A, or T nucleotide or G, A, or C nucleotide respectively. In particular embodiments, the PCR primer is at least or no more than 10, 11, 12, 13, 14, 15, 16, 17, 18, 19, 20, 21, 22, 23, 24, 25, 26, 27, 28, 29, 30, 31, 32, 33, 34,3 5, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, or more nucleotides in length with 100% homology to the template sequence, with the potential exception of non-homology the SNP location. After several rounds of amplifications, if the PCR primers generate the expected band size, the SNP can be determined to have the A nucleotide and not the G nucleotide.

### B. Copy Number Variation Detection

Particular embodiments of the disclosure concern methods of detecting a copy number variation (CNV) of a particular allele. One can utilize any known method for detecting CNVs to detect the CNVs. Such methods include fluorescent in situ hybridization, comparative genomic hybridization, arrays, polymerase chain reaction, sequencing, or a combination thereof, for example. In some embodiments, the CNV is detected using an array. Array platforms such as those from Agilent, Illumina, or Affymetrix may be used, or custom arrays could be designed. One example of how an array may be used includes methods that comprise one or more of the steps of isolating nucleic acid material in a suitable manner from an organism suspected of having the CNV and, at least in some cases from an organism or reference genome that does not have the CNV; processing the nucleic acid material by fragmentation, labelling the nucleic acid with, for example, fluorescent labels, and purifying the fragmented and labeled nucleic acid material; hybridizing the nucleic acid material to the array for a sufficient time, such as for at least 24 hours; washing the array after hybridization; scanning the array using an array scanner; and analyzing the array using suitable software. The software may be used to compare the nucleic acid material from the organism suspected of having the CNV to the nucleic acid material of an organism who is known not to have the CNV or a reference genome.

In some embodiments, detection of a CNV is achieved by polymerase chain reaction (PCR). PCR primers can be employed to amplify nucleic acid at or near the CNV wherein an organism with a CNV will result in measurable higher levels of PCR product when compared to a PCR product from a reference genome. The detection of PCR product amounts could be measured by quantitative PCR (qPCR) or could be measured by gel electrophoresis, as examples. Quantification using gel electrophoresis comprises subjecting the resulting PCR product, along with nucleic acid standards of known size, to an electrical current on an agarose gel and measuring the size and intensity of the resulting band. The size of the resulting band can be compared to the known standards to determine the size of the resulting band. In some embodiments, the amplification of the CNV will result in a band that has a larger size than a band that is amplified, using the same primers as were used to detect the CNV, from a reference genome or an organism that does not have the CNV being detected. The resulting band from the CNV amplification may be nearly double, double, or more than double the resulting band from the reference genome or the resulting band from an organism that does not have the CNV being detected. In some embodiments, the CNV can be detected using nucleic acid sequencing. Sequencing techniques that could be used include, but are not limited to, whole genome sequencing, whole exome sequencing, and/or targeted sequencing.

### C. DNA Sequencing

In some embodiments, DNA may be analyzed by sequencing. The DNA may be prepared for sequencing by any method known in the art, such as library preparation, hybrid capture, sample quality control, product-utilized ligation-based library preparation, or a combination thereof. The DNA may be prepared for any sequencing technique. In some embodiments, a unique genetic readout for each sample may be generated by genotyping one or more highly polymorphic SNPs. In some embodiments, sequencing, such as 76 base pair, paired-end sequencing, may be performed to cover approximately 70%, 75%, 80%, 85%, 90%, 95%, 99%, or greater percentage of targets at more than 20×, 25×, 30×, 35×, 40×, 45×, 50×, or greater than 50× coverage. In certain embodiments, mutations, SNPS, INDELS, copy number alterations (somatic and/or germline), or other genetic differences may be identified from the sequencing using at least one bioinformatics tool, including VarScan2, any R package (including CopywriteR) and/or Annovar.

### D. RNA Sequencing

In some embodiments, RNA may be analyzed by sequencing. The RNA may be prepared for sequencing by any method known in the art, such as poly-A selection, cDNA synthesis, stranded or nonstranded library preparation, or a combination thereof. The RNA may be prepared for any type of RNA sequencing technique, including stranded specific RNA sequencing. In some embodiments, sequencing may be performed to generate approximately 10M, 15M, 20M, 25M, 30M, 35M, 40M or more reads, including paired reads. The sequencing may be performed at a read length of approximately 50 bp, 55 bp, 60 bp, 65 bp, 70 bp, 75 bp, 80 bp, 85 bp, 90 bp, 95 bp, 100 bp, 105 bp, 110 bp, or longer. In some embodiments, raw sequencing data may be converted to estimated read counts (RSEM), fragments per kilobase of transcript per million mapped reads (FPKM), and/or reads per kilobase of transcript per million mapped reads (RPKM). In some embodiments, one or more bioinformatics tools may be used to infer stroma content, immune infiltration, and/or tumor immune cell profiles, such as by using upper quartile normalized RSEM data.

### E. Proteomics

In some embodiments, protein may be analyzed by mass spectrometry. The protein may be prepared for mass spectrometry using any method known in the art. Protein, including any isolated protein encompassed herein, may be treated with DTT followed by iodoacetamide. The protein may be incubated with at least one peptidase, including an endopeptidase, proteinase, protease, or any enzyme that cleaves proteins. In some embodiments, protein is incubated with the endopeptidase, LysC and/or trypsin. The protein may be incubated with one or more protein cleaving enzymes at any ratio, including a ratio of μg of enzyme to μg protein at approximately 1:1000, 1:100, 1:90, 1:80, 1:70, 1:60, 1:50, 1:40, 1:30, 1:20, 1:10, 1:1, or any range between. In some embodiments, the cleaved proteins may be purified, such as by column purification. In certain embodiments, purified peptides may be snap-frozen and/or dried, such as dried under vacuum. In some embodiments, the purified peptides may be fractionated, such as by reverse phase chromatography or basic reverse phase chromatography. Fractions may be combined for practice of the methods of the disclosure. In some embodiments, one or more fractions, including the combined fractions, are subject to phosphopeptide enrichment, including phospho-enrichment by affinity chromatography and/or binding, ion exchange chromatography, chemical derivatization, immunoprecipitation, co-precipitation, or a combination thereof. The entirety or a portion of one or more fractions, including the combined fractions and/or phospho-enriched fractions, may be subject to mass spectrometry. In some embodiments, the raw mass spectrometry data may be processed and normalized using at least one relevant bioinformatics tool.

### II. Proteins

As used herein, a “protein” or “polypeptide” refers to a molecule comprising at least five amino acid residues. As used herein, a “peptide” refers to a molecule comprising at least three amino acid residues. As used herein, the term “wild-type” refers to the endogenous version of a molecule that occurs naturally in an organism. In some embodiments, wild-type versions of a protein or polypeptide are employed, however, in many embodiments of the disclosure, a modified protein or polypeptide is employed. The terms described above may be used interchangeably. A “modified protein” or “modified polypeptide” or a “variant” refers to a protein or polypeptide whose chemical structure, particularly its amino acid sequence, is altered with respect to the wild-type protein or polypeptide. In some embodiments, a modified/variant protein or polypeptide has at least one modified activity or function (recognizing that proteins or polypeptides may have multiple activities or functions). It is specifically contemplated that a modified/variant protein or polypeptide may be altered with respect to one activity or function yet retain a wild-type activity or function in other respects, such as immunogenicity.

Where a protein is specifically mentioned herein, it is in general a reference to a native (wild-type) or recombinant (modified) protein or, optionally, a protein in which any signal sequence has been removed. The protein may be isolated directly from the organism of which it is native, produced by recombinant DNA/exogenous expression methods, or produced by solid-phase peptide synthesis (SPPS) or other in vitro methods. In particular embodiments, there are isolated nucleic acid segments and recombinant vectors incorporating nucleic acid sequences that encode a polypeptide. The term “recombinant” may be used in conjunction with a polypeptide or the name of a specific polypeptide, and this generally refers to a polypeptide produced from a nucleic acid molecule that has been manipulated in vitro or that is a replication product of such a molecule.

In certain embodiments the size of a protein or polypeptide (wild-type or modified) may comprise, but is not limited to, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1100, 1200, 1300, 1400, 1500, 1750, 2000, 2250, 2500 amino acid residues or greater, and any range derivable therein, or derivative of a corresponding amino sequence described or referenced herein. It is contemplated that polypeptides may be mutated by truncation, rendering them shorter than their corresponding wild-type form, also, they might be altered by fusing or conjugating a heterologous protein or polypeptide sequence with a particular function. As used herein, the term “domain” (also “protein domain”) refers to any distinct functional or structural unit of a protein or polypeptide, and generally refers to a sequence of amino acids with a structure or function recognizable by one skilled in the art.

The nucleotide as well as the protein, polypeptide, and peptide sequences for various genes have been previously disclosed, and may be found in the recognized computerized databases. Two commonly used databases are the National Center for Biotechnology Information's Genbank and GenPept databases (on the World Wide Web at ncbi.nlm.nih.gov/) and The Universal Protein Resource (UniProt; on the World Wide Web at uniprot.org). The coding regions for these genes may be amplified and/or expressed using the techniques disclosed herein or as would be known to those of ordinary skill in the art.

It is contemplated that in compositions of the disclosure, there is between about 0.001 mg and about 10 mg of total polypeptide, peptide, and/or protein per ml. The concentration of protein in a composition can be about, at least about or at most about 0.001, 0.010, 0.050, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0 mg/ml or more (or any range derivable therein).

### EXAMPLES

The following examples are included to demonstrate certain embodiments of the invention. It should be appreciated by those of skill in the art that the techniques disclosed in the examples which follow represent techniques discovered by the inventor to function well in the practice of the invention, and thus can be considered to constitute certain modes for its practice. However, those of skill in the art should, in light of the present disclosure, appreciate that many changes can be made in the specific embodiments which are disclosed and still obtain a like or similar result without departing from the spirit and scope of the invention.

### Example 1

### Defining Hierarchical Protein Interaction Networks from Spectral Analysis of Bacterial Proteomes

Cellular phenotypes emerge from a hierarchy of molecular interactions: proteins interact to form complexes, pathways, and phenotypes. The inventors show that hierarchical networks of protein interactions can be extracted from the statistical pattern of proteome variation as measured across thousands of bacteria and that these hierarchies reflect the emergence of complex bacterial phenotypes. The inventors describe the mathematics underlying the statistical approach and validate the results through gene-set enrichment analysis and comparison to existing experimentally-derived hierarchical databases. The inventors demonstrate the biological utility of the unbiased hierarchical models by creating a model of motility in Pseudomonas aeruginosa and using it to discover a previously unappreciated genetic effector of twitch-based motility. Overall, the disclosed approach, SCALES (Spectral Correlation Analysis of Layered Evolutionary Signals), predicts hierarchies of protein interaction networks describing emergent biological function using only the statistical pattern of bacterial proteome variation.

The inventors hypothesized that (i) pairwise and higher-order information could be directly extracted from the statistical pattern of covariation between orthologs and (ii) this information could then be used to create a single multi-scale hierarchical model describing the emergence of complex phenotypes from individual proteins. The inventors used Singular Value Decomposition (SVD) to spectrally analyze a large database of non-redundant bacterial proteomes and to define a set of components of ortholog covariation (an ‘SVD spectrum’). The inventors found that covariation described by the SVD spectrum was distributed according to biological scale: top components were enriched for phylogenetic information, deeper components for higher-order protein interactions resembling pathways (‘indirect’ interactions), and deepest components for pairwise PPIs resembling physically interacting protein complexes (‘direct’ interactions). Second, the inventors introduced the concept of ‘spectral correlations’, a metric representing the extent to which two bacteria or proteins share a similar statistical pattern of covariation within a specific region of the SVD spectrum. The inventors found that machine-learning models trained on spectral correlation features could simultaneously predict indirect and direct PPIs with higher-fidelity relative to existing computational and experimental methods. Third, the inventors introduced the concept of ‘spectral depth’—a way to relate spectral correlations between different positions in the SVD spectrum. Serially thresholding spectral depth defined hierarchically related protein interaction networks. The inventors found the statistically derived hierarchies reflect the emergence of complex cellular phenotypes in bacteria as evidenced by gene-set enrichment analysis (GSEA) and comparison with experimentally derived hierarchies in the Kyoto Encyclopedia of Genes and Genomes (KEGG). The topology of these hierarchies were that bottom layers define protein interaction networks representing specific functions of protein complexes, middle layers define the integration of networks within bottom layers into broader functions resembling pathways, and top layers define the integration of networks in middle layers into high-level functions resembling phenotypes. Finally, the inventors showed the utility of the approach by assigning global and local functions to an uncharacterized protein in Pseudomonas aeruginosa and validating these predictions experimentally.

### Results

Spectral Decomposition of Orthologous Gene Content Among Bacteria Organizes Covariation from Phylogenetic Relationships Down to Pairwise Ppis

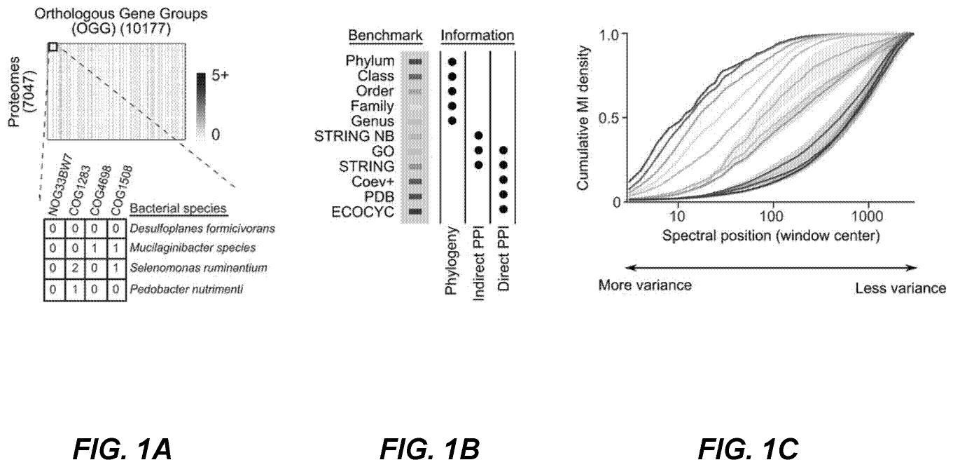

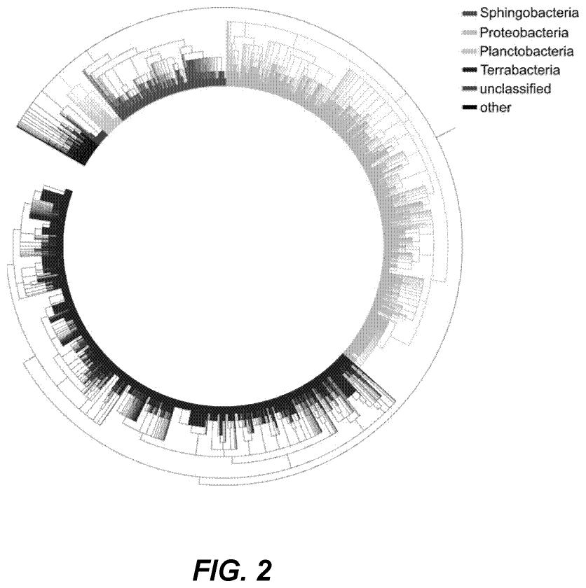

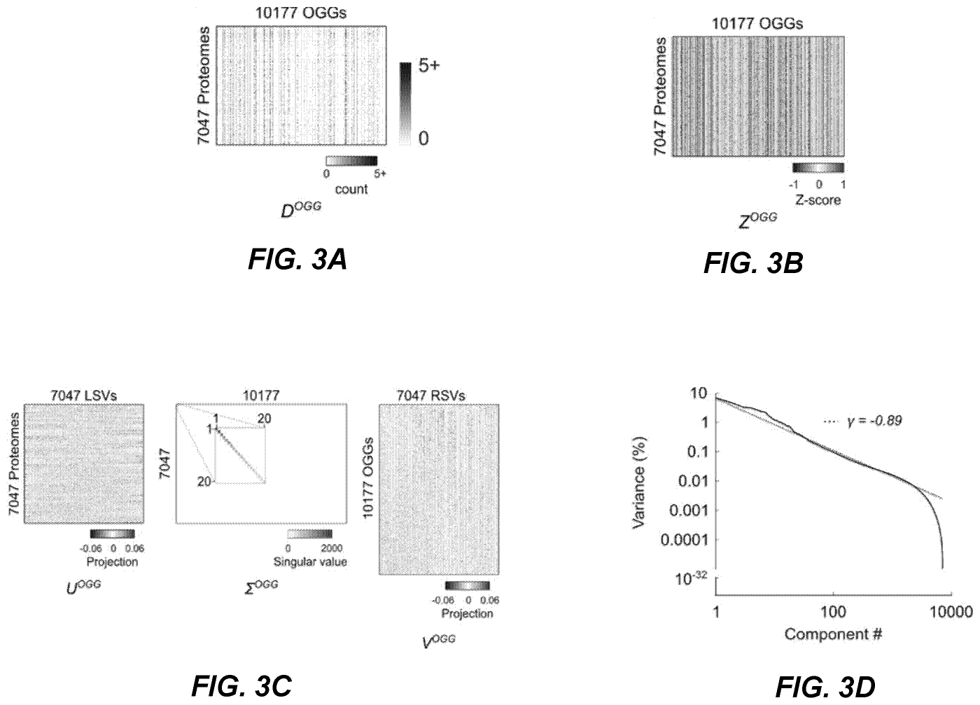

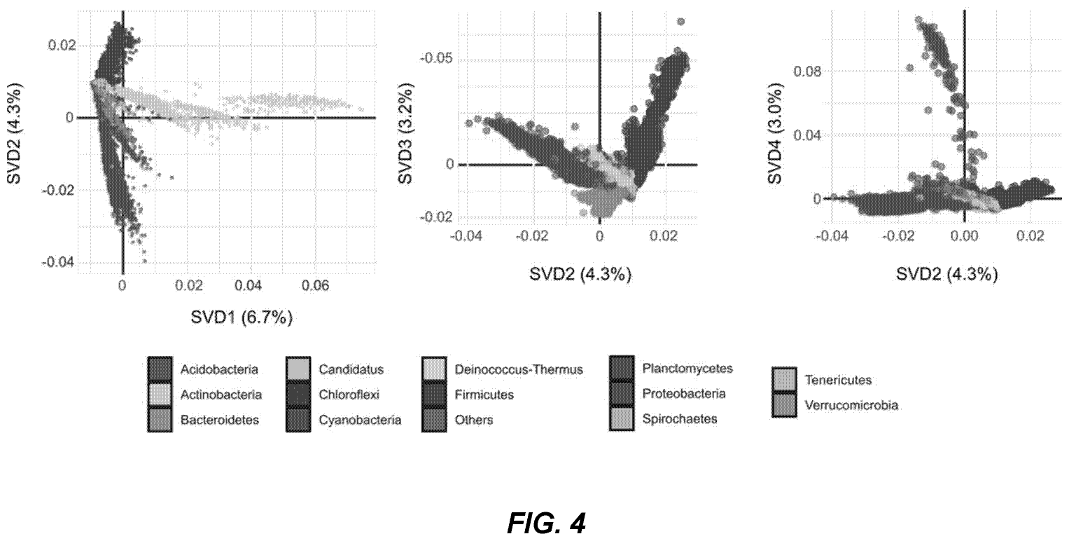

Orthologs are genes in different species that originated from a common ancestral gene and typically share a conserved core function. Assignment of orthologous gene groups (OGGs) is a robust and computationally tractable heuristic method for inferring orthologs and has been used extensively in comparative genomics (Overbeek et al., 1999). To sample variation in the OGG content of bacterial proteomes, the inventors created a matrix, DOGG, where each row is one of 7,047 UniProt bacterial reference proteomes, each column is one of 10,177 OGGs, and each entry is the number of times an OGG was observed in a proteome (FIG. 1A, FIG. 2, Materials and Methods) (The UniProt Consortium, 2019; Huerta-Cepas et al., 2017; Huerta-Cepas et al., 2019). The inventors spectrally decomposed DOGG using SVD (Materials and Methods) (Klema and Laub, 1980) (FIGS. 3A-3C). SVD reveals patterns of correlations within the data by defining components of covariation and ordering them by their ability to explain the total data variance: SVD component 1 (SVD1) explains more data-variance than SVD2, SVD2 more than SVD3, etc (FIG. 3D). The inventors observed that rows of DOGG corresponding to organisms sharing the highest level of phylogenetic classification, i.e. phylum, clustered together when projected onto SVD1, SVD2, SVD3, or SVD4 suggesting that the most dominant patterns of OGG covariation arise from global phylogenetic relationships (FIG. 4). The vast majority of the overall data variance (83%) was not explained by by SVD1, SVD2, SVD3, and SVD4 taken together. The inventors next sought to systematically interrogate what biological information, if any, exists amongst deeper regions of the SVD spectrum of DOGG.

To quantify biological information contained within different regions of the SVD spectrum, the inventors computed the mutual information (MI) shared between known biological relationships spanning phylogeny to pairwise PPIs and the proximity between two proteomes or proteins as defined statistically by the SVD spectrum. A high MI value indicates that the statistical proximity reflects the known biological relationship, just as the clustering of proteomes on SVD1, SVD2, SVD3, and SVD4 reflected phylum classification (FIG. 4) In the following paragraphs the inventors detail how the inventors defined benchmarks of known biological relationships and statistical proximity between proteomes or proteins within a region of the SVD spectrum.

Benchmarks were assembled using existing biological databases to represent a hierarchy of organization from phylogenetic classification to indirect interactions in cellular pathways and to direct PPIs in protein complexes (FIG. 1B, Materials and Methods). The NCBI taxonomy database was used to assemble five different phylogenetic benchmarks indicating if two bacteria share the same taxonomic substrings down to the levels of phylum, class, order, family or genus (NCBI Resource Coordinators, 2018). Pathway level benchmarks were assembled by mining indirect PPIs found in the STRING or GO databases (Szklarczyk et al., 2018; The Gene Ontology Consortium, 2020). Finally, protein complex benchmarks were assembled by incorporating direct PPIs identified in the Protein Databank (PDB), ECOCYC database, or by analyzing amino-acid level coevolution (Coev+) (Kesler et al., 2016; Cong et al., 2019). The inventors focused the analyses on E. coli K12, a well-studied model organism for which a large number of high quality, experimentally supported PPIs are known.

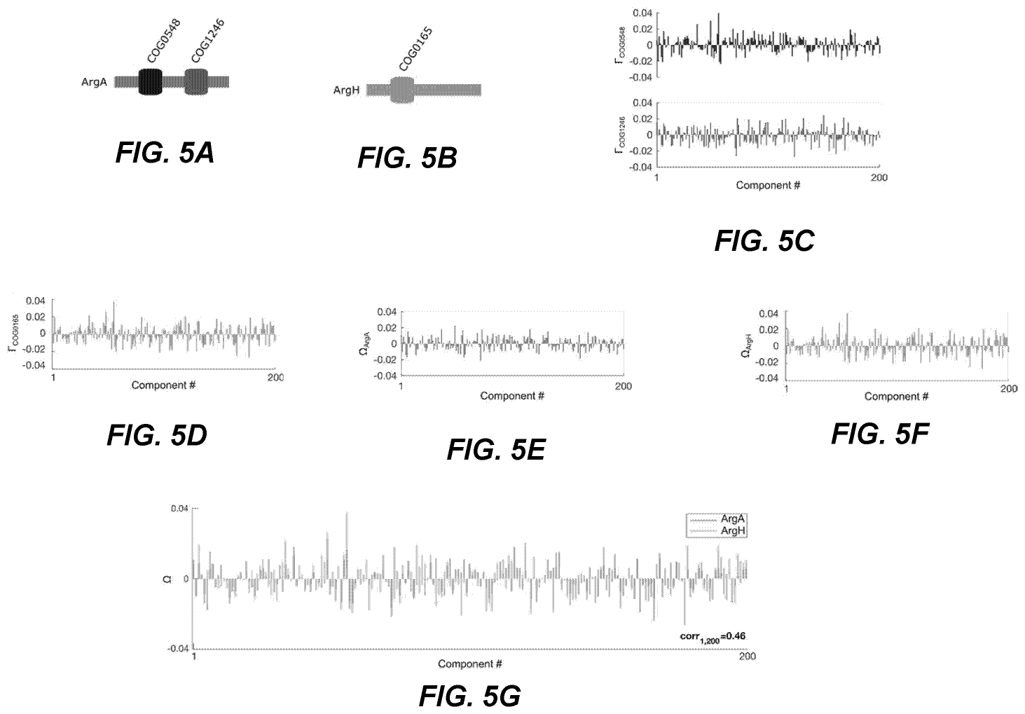

The inventors quantified proximity within a specific region of the SVD spectrum of D^{OGG} by introducing a metric the inventors term ‘spectral correlation’ (Materials and Methods). In detail, application of SVD to D^{OGG} produces two projection matrices U^{OGG} and V^{OGG} (FIGS. 3A-3C). A row of U^{OGG} contains the projections of a proteome onto each SVD component: column one onto SVD1, column two onto SVD2, and so on. Similarly, the rows of V^{OGG} contain the projections of an OGG onto each SVD component. Spectral correlations are the Pearson correlations between two rows of U^{OGG} or V^{OGG}. To compute spectral correlations for a specific region of the SVD spectrum, Pearson correlations are computed across the columns of U^{OGG} or V^{OGG} representing only the components of interest. The interpretation of a positive spectral correlation is that the two proteomes or OGGs are proximal when projected onto the specified set of SVD components. Because a single protein-coding gene can have multiple OGGs, the inventors approximated the projection of a protein onto each SVD component by averaging the proteins' constituent OGG projections and then computed protein-protein spectral correlations as above (FIG. 5).

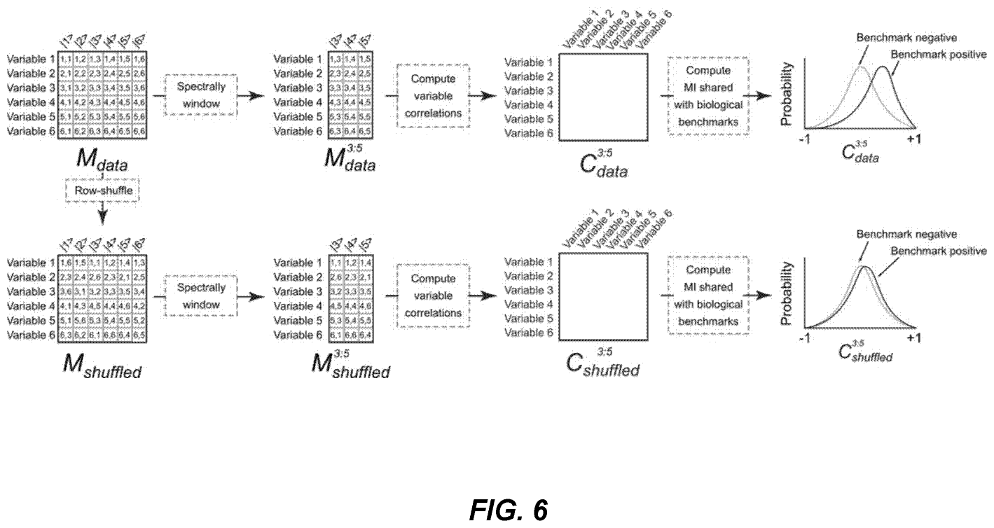

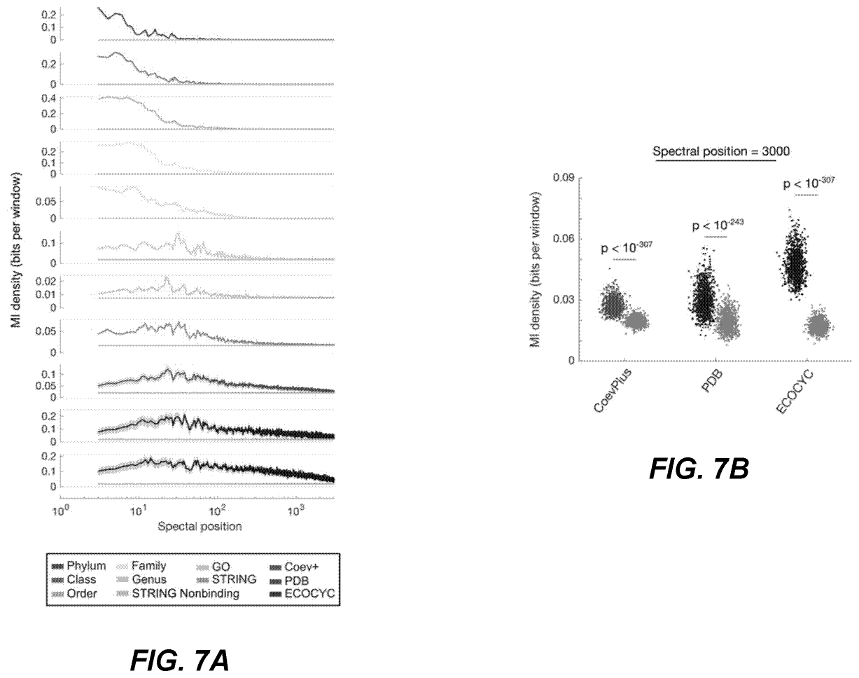

The inventors computed the MI shared between each benchmark and spectral correlations within all five-component windows of the SVD spectrum of D^{OGG} (Materials and Methods). To estimate contributions of spurious correlations arising from finite sampling and overlap in OGG structure between proteins the inventors computed the MI for a randomized projection matrix with bootstrap support (FIG. 6). The inventors observed that the MI density (bits per window) for phylogenetic benchmarks declined rapidly as the spectral window was shifted deeper into the SVD spectrum, quickly converging upon the MI produced by spurious correlations (FIG. 7A). In contrast, the MI density decayed more slowly with spectral position for benchmarks of indirect PPIs and most slowly for benchmarks of direct PPIs. In fact, SVD2995-3000 harbored significantly greater direct PPI MI than that produced by the model of spurious correlations despite accounting for only 0.021% of data-variance (p<10⁻²⁴³ by pairwise Student's T-test, FIG. 7B). These results suggest that the first 3000 components of the SVD spectrum of D^{OGG} contain meaningful biological information.

The inventors computed MI cumulative distribution functions (MI cdfs) for each benchmark across the top 3000 SVD components after subtracting the contributions of spurious correlations (Materials and Methods). Qualitatively, the MI cdf is the relative amount of MI gained as a function of depth; reaching a value of ‘1’ indicates that deeper regions hold no more biologically meaningful information regarding a benchmark. The inventors observed that the MI cdfs for different types of benchmarks approached one in the following order: phylum, class, order, family, genus, indirect PPIs, mixed indirect/direct PPIs, and direct PPIs (FIG. 1C). Of note, the MI cdfs for the three different benchmarks of direct PPIs—ECOCYC, PDB, and Coev+—were nearly superimposable, suggesting that the MI estimates were robust to benchmark source. Taken together, these results demonstrated that spectral decomposition of bacterial OGG variation using SVD organizes covariation from phylogenetic relationships down to pairwise PPIs.

### Using Spectrally Resolved Covariation to Train Random Forest Models to Predict Indirect and Direct PPIs in E. Coli K12

A well-known challenge of using covariation to infer PPIs is that protein covariation can represent phylogenetic relationships, indirect interactions in pathways, direct interactions in protein complexes, or noise (Schafer and Strimmer, 2005; Sul et al., 2018; Nagy et al., 2020). The results above demonstrated that the SVD spectrum of DOGG separates covariation arising from these different sources. Therefore, the inventors hypothesized that spectral correlations measured across specific SVD components could be used to accurately predict PPIs and assign them to a scale of organization, i.e. indirect vs direct PPI.

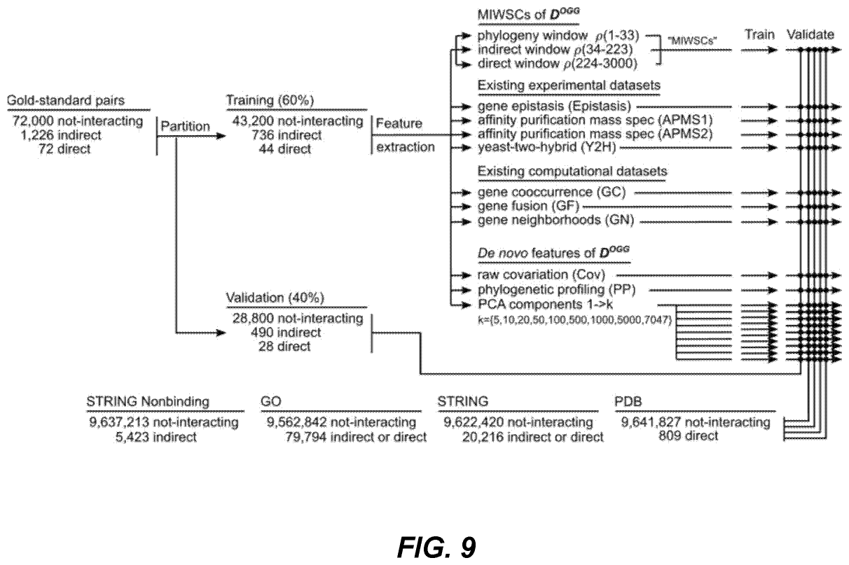

To test this hypothesis, the inventors devised a multi-level classification task where a machine learning algorithm was challenged to classify pairs of E. coli K12 proteins as not-interacting, indirect PPI, or direct PPI (FIG. 9, Materials and Methods). For model training and validation, a gold-standard set of well characterized E. coli K12 protein pairs was assembled: 72,000 not-interacting pairs, 1,226 indirect PPIs, 72 direct PPIs. The indirect and direct PPIs were stringently chosen based on representation in multiple databases to reduce the rate of false positives in individual databases (Rajagopala et al., 2014). The not-interacting pairs were chosen by random selection. The relative numbers of not-interacting, indirect PPIs, and direct PPIs were chosen based on prior estimates of the true proportions of these interaction classes in biology (Rajagopala et al., 2014; Cong et al., 2019)The gold-standard dataset was randomly partitioned into training (60%) and validation (40%) sets. As will be described in detail below, spectral correlation features and various comparison features were extracted for the protein pairs in the gold-standard dataset. For each feature, Random Forest (RF) models were trained using labeled examples in the training dataset and validated using unlabeled examples from the validation dataset. Additional validation tasks performed included predicting out-of-bag examples in the training dataset and predicting examples in four additional comprehensive benchmarks (STRING Nonbinding: n=14,793 indirect PPIs, GO: n=79,794 indirect or direct PPIs, STRING: n=14,793 indirect PPIs and n=5,423 direct PPIs, PDB: n=809 direct PPIs). RF model performance was quantified by computing F-scores for the predictions of each interaction class. F-score is the harmonic mean of precision and recall providing a holistic assessment of both the accuracy and completeness of each class prediction. Finally, the entire process of randomly partitioning the gold-standard dataset, training, and validation was repeated 50 times to assess the reproducibility of the resultant model performance.

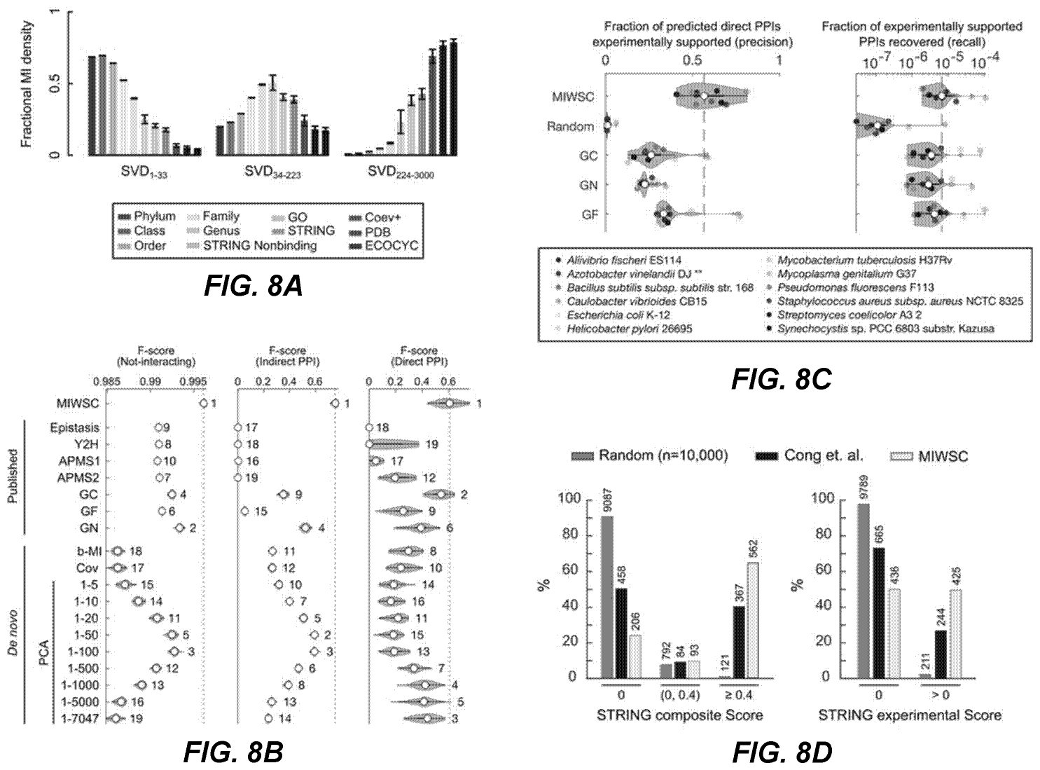

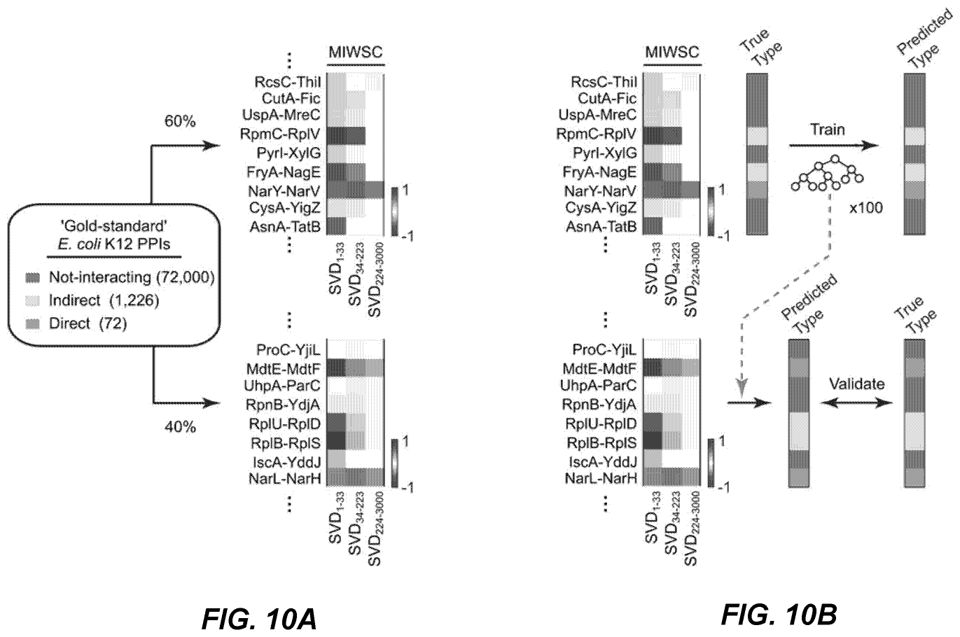

To define spectral correlation features the inventors computed spectral correlations across three sets of SVD components. The selection of these three sets was informed by the results of FIG. 1C: SVD1-33 spanned up to the 25^{th} percentile point of the STRING nonbinding MI cdf, SVD34-223 spanned the 25^{th} to 75^{th} percentile points of the STRING nonbinding MI cdf, and SVD224-3000 spanned the 75^{th} percentile point to the point at which MI estimates converged upon that of the model of spurious correlations. SVD1-33, SVD34-223, SVD234-3000 isolated the majority of the MI related to either phylogeny, indirect PPIs, or direct PPIs respectively (FIG. 8A). For each pair of proteins in the gold standard dataset, the inventors computed spectral correlations across each of these three sets of SVD components (FIG. 10A). The inventors term this set of three correlation values as the ‘MI windowed spectral correlations’ (MIWSCs) feature.

Comparison features were extracted from various existing datasets derived using established methods of PPI inference. These included the experimental methods of yeast-two-hybrid (‘Y2H’), affinity-purification mass spectrometry (APMS1’, ‘APMS2’) and gene epistasis (‘epistasis’), as well as the computational methods of phylogeny-aware gene co-occurrence (‘GC’), gene neighborhood (‘GN’), and gene fusion (‘GF’) (Szklarczyk et al., 2019; Rajagopala et al., 2014; Babu et al., 2014; Babu et al., 2018; Hu et al., 2009). As differences in the size and quality of the underlying dataset can influence the fidelity of computational PPI inference, the inventors extracted additional comparison features directly from the dataset (D^{OGG}). These additional features included binary MI (‘b-MI’), raw covariation (‘Cov’), and a Principal Components Analysis (PCA) based approach considering the top k SVD components (SVD1-k) (Materials and Methods) (Pellegrini et al., 1999; Franceschini et al., 2016).

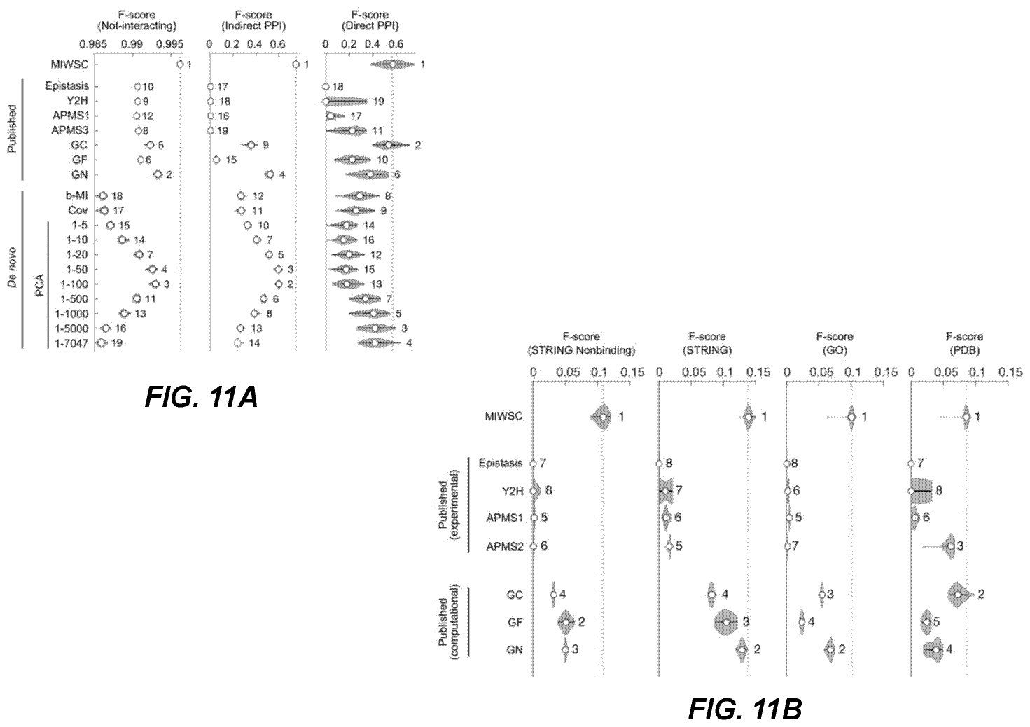

In the multiscale classification task, the RF models trained on MIWSCs produced significantly greater F-scores across all three interaction classes compared to models trained on any of the other 18 different features in all validation tasks (FIG. 8B, FIG. 11, statistical comparisons by Wilcoxon rank-sum test).

With regard to the PCA based approach, models trained on SVD1-5 performed at or below the median rank of all models across all three classes. F-scores for predicting indirect PPIs increased as components up to SVD100 were included (SVD1-100) without improvement of the prediction of direct PPIs. Including components beyond SVD100 increased the F-scores for predicting direct PPIs while decreasing F-scores for predicting indirect PPIs. Thus, the PCA based approach may not be ideal because models trained on the PCA-based features poorly predicted indirect PPIs, direct PPIs, or both. Taken together, these data show that the MIWSCs feature is the only one of the tested features that informs high fidelity PPI predictions in E. coli K12 across multiple scales of biological organization.

### Rf Models Trained to Predict PPIs in E. Coli K12 Using Miwsc'S Generalize Across Diverse Bacteria

To test generalizability of the RF models trained on MIWSCs in E. coli K12 to other organisms the inventors predicted proteome-wide direct PPIs for 11 additional phylogenetically diverse bacteria, including one organism (Azotobacter vinelandii) that was not represented in D^{OGG} (Materials and Methods). For comparison, the inventors predicted direct PPIs for the same organisms using either random selection (‘Random’, n=10,000) or by mining the high confidence interactions (confidence score >0.7) in the STRING subchannels for the methods of GC, GN, or GF. To benchmark against orthogonal experimental evidence, the inventors mined the experimental subchannel of the STRING database (Materials and Methods).

The median precision (5^{th}-95^{th} percentile range in parenthesis) was significantly greater for direct PPIs predicted by the RF models trained on MIWSCs: 56.6% (41.0-81.2), 0.9% (0.6-5.7), 26.1% (13.3-56.9), 22.4% (18.2-34.1), or 33.6% (29.5-74.6) for MIWSC RF models, random selection, GC, GN, or GF respectively (FIG. 8C, left panel, one-sided Wilcoxon rank sum test with multiple comparison). In addition, the median recall was significantly greater for RF models trained on MIWSCs (FIG. 8C, right panel). The recall values were low across all methods, which may reflect the previously reported high number of false-positives in experimental databases (Rajagopala et al., 2014; Cong et al., 2019). Nevertheless, the MIWSC RF models predicted a median (IQR in parenthesis) of 897 (551 to 1609) direct PPIs per proteome.

In addition, the inventors performed a head-to-head comparison for predicting direct PPIs in Mycobacterium tuberculosis H37Rv using the MIWSC RF models versus the method of Cong and colleagues that infers direct PPIs from proteome-wide amino acid coevolution (Materials and Methods) (Cong et al., 2019). The inventors found that the MIWSC RF models exhibited a significantly greater precision and recall when benchmarked against the STRING composite score, as done previously by Cong and colleagues, or against the STRING experimental scores (FIG. 8D, Chi-squared test).

Taken together, these results suggested that the RF models trained to predict E. coli K12 PPIs using MIWSCs were robust and generalizable across diverse bacteria.

### Spectral Decomposition of Protein Domain Content Organizes Covariation According to Biological Scale and Informs Accurate Indirect and Direct PPI Predictions

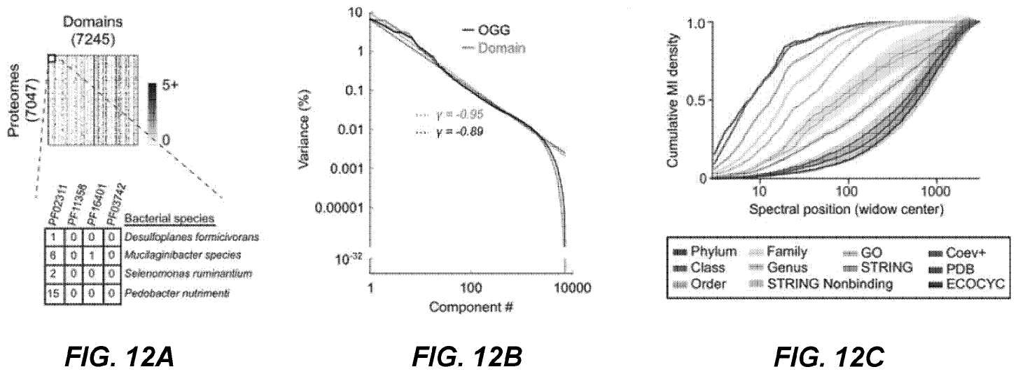

The inventors sought to test whether the results the inventors observed above were specific to choosing OGGs as a feature of orthology. Protein domains are conserved parts of proteins that have been previously used as a feature of bacterial orthology (Mistry and Finn, 2007). The inventors defined a new matrix, D^{domain}, where each row is one of the 7,047 proteomes used in the D^{OGG} matrix, each column is one of 7,245 unique protein domains, and each entry is the number of times a domain appears in a proteome (FIG. 12A).

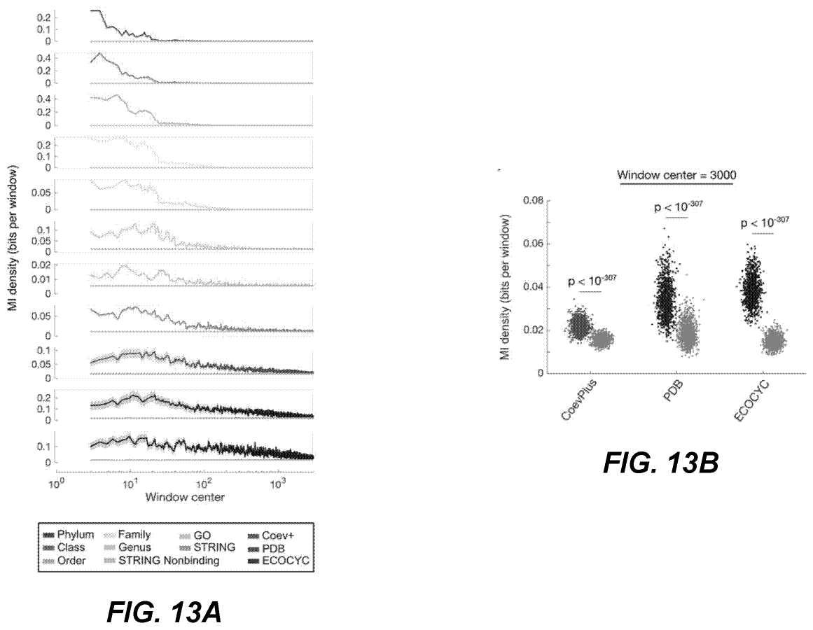

The SVD spectrum derived from D)domain was nearly superimposable on that of D^{OGG}, suggesting that the statistical structure of covariation is similar across these different orthologous features (FIG. 12B, Materials and Methods). Similar to the analysis of D^{OGG} described above, the inventors quantified the MI shared between the various benchmarks of prior biological knowledge and spectral correlations within all 5 component windows of the SVD spectrum of D^{domain}. Again, the inventors observed that the MI density (bits per window) for phylogenetic benchmarks declined rapidly as the spectral window was shifted deeper into the SVD spectrum (FIG. 13A). In contrast, the MI for indirect PPI benchmarks declined more slowly and the MI for direct PPI benchmarks remained statistically significant until at least SVD3000 (Paired Student's T-test see FIGS. 13A-13B). MI cdfs were computed for each benchmark and found to mirror those derived for DOGG: ordered according to phylum, class, order, family, genus, indirect PPI, mixed indirect/direct PPI, direct PPI (FIG. 12C, Materials and Methods).

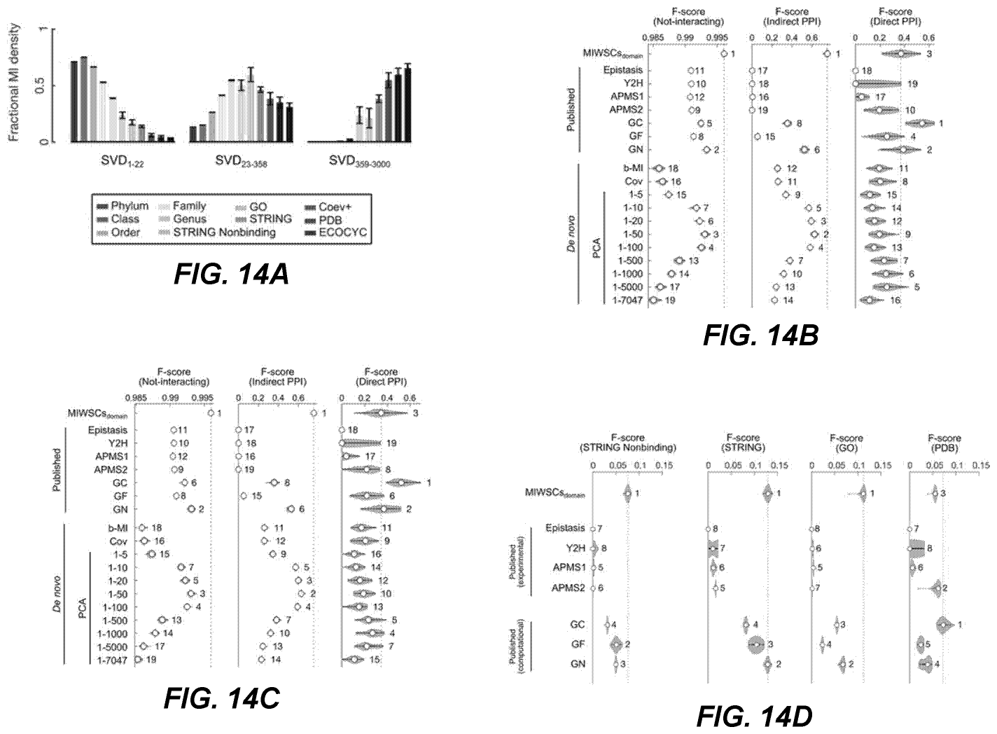

RF models were trained to predict PPIs in E. coli K12 using MIWSCs computed from the SVD spectrum of D^{domain} (MIWSCsdomain). These models were validated in the same multilevel classification tasks as described above for D^{OGG} (FIG. 14). When compared to RF models trained on features of existing computational and experimental methods, the RF models trained on MIWSCsdomain ranking 1st, 1st, and 3rd for the classes of not-interacting, indirect PPI, and direct PPI respectively.

Taken together, these results illustrate that spectral decomposition of orthologous gene content across bacterial proteomes separates covariation arising from different biological scales regardless of whether orthology is defined through orthologous gene groups or protein domains. As a result of this spectral separation, spectral correlations computed across sets of SVD components of OGG or domain covariation can produce accurate predictions of PPIs at different biological scale, i.e. indirect PPIs and direct PPIs.

### A Statistically-Defined Hierarchy of Protein Interaction Networks Describing the Emergent Phenotype of Directed Motility in E. Coli K12

Understanding the molecular basis of a phenotype requires (i) identifying units of collective function at different biological scales and (ii) relating these scales to create a hierarchical model of how a phenotype emerges from a set of proteins. A useful example is the experimentally derived hierarchical model of directed motility in E. coli K12 (KEGG hierarchy, BRITE ECO:02035). At the lowest levels in this hierarchy, physical interactions between proteins create small units of collective structure and function, such as a basal body, rod, ring, motor, and filament. Integration of these structures and their individual functions produces the flagellum, a machine that turns to move the cell. Integration of the flagellum and the chemotaxis system ultimately produces directed motility—the ability to move purposefully along a chemical gradient.

The inventors hypothesized that the inventors could derive a multiscale, hierarchical model of this phenotype in a purely data-driven and unbiased manner using only the SVD spectrum of D^{OGG}. To do so, the inventors first developed a model of spectral correlations between non-interacting proteins. The inventors then applied this model to identify ‘significant’ protein correlations within different regions of the SVD spectrum. These significant proteins correlations represented statistically predicted protein interactions. Next, the inventors defined a metric termed ‘spectral depth’ as the deepest spectral position to which two proteins remained significantly correlated. The inventors posited that applying serial thresholds to spectral depth would identify a tree-like hierarchy where the root of the tree is the protein interaction network observed at shallower spectral depth thresholds and the branches are the networks defined at deeper spectral depth thresholds. The details of creating the model of non-interacting protein correlations and defining a hierarchy using spectral depth are detailed below.

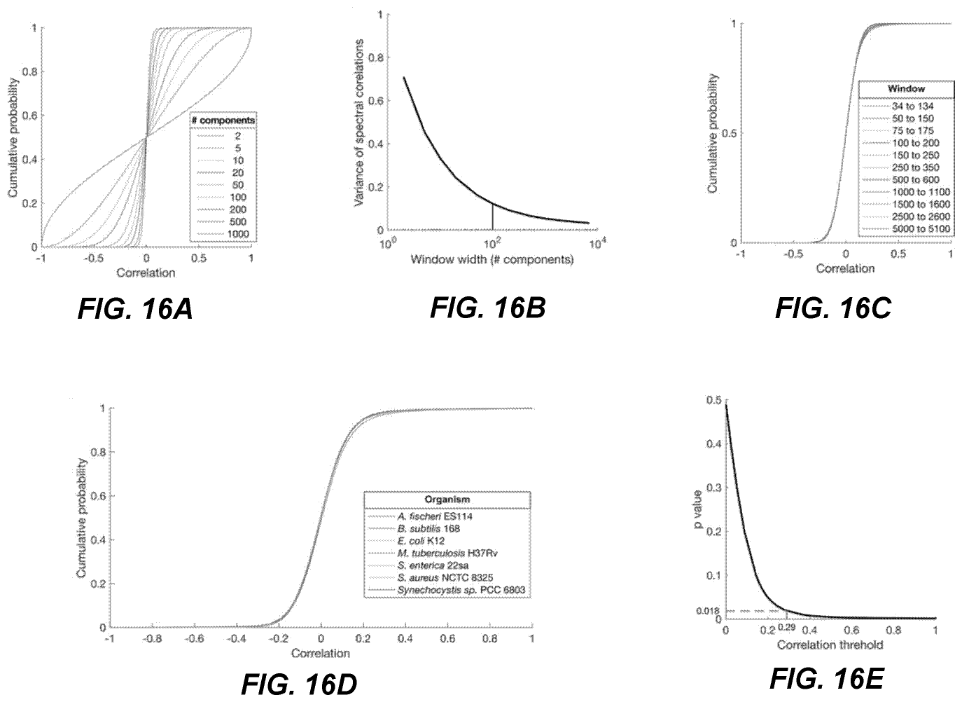

To define a model of spectral correlations between non-interacting proteins, the inventors first considered the distribution of all pairwise spectral correlations centered on SVD 1000 for the proteins encoded in the proteome of E. coli K12. the rationale was that since the vast majority of proteins do not interact to produce a collective function, the distribution of all-by-all spectral correlations approximates that of correlations between proteins that do not functionally interact. The inventors observed that the variance of this distribution decreased rapidly as the correlation window widened until about a 100 component width—motivating the choice of computing correlations over sets of 100 components (FIGS. 16A-16B). The inventors computed distributions of all-by-all correlations between E. coli K12 proteins across windows centered on different regions of the SVD spectrum and found them to be superimposable (FIG. 16C). Additionally, the inventors computed such distributions for proteins from other diverse bacteria and found them to be superimposable with those derived from E. coli K12 (FIG. 16D). These properties enabled the inventors to define a constant threshold for significant spectral correlations between two proteins across any 100 component SVD window. The p-value derived from the empirical CDF of this model decreased rapidly until a threshold value of 0.29 (FIG. 16E). Therefore, the inventors chose the value of 0.29 associated with a p-value of 0.018 as the threshold of spectral correlations signifying functional interactions between proteins derived from any bacterial proteome within any region of the SVD spectrum.

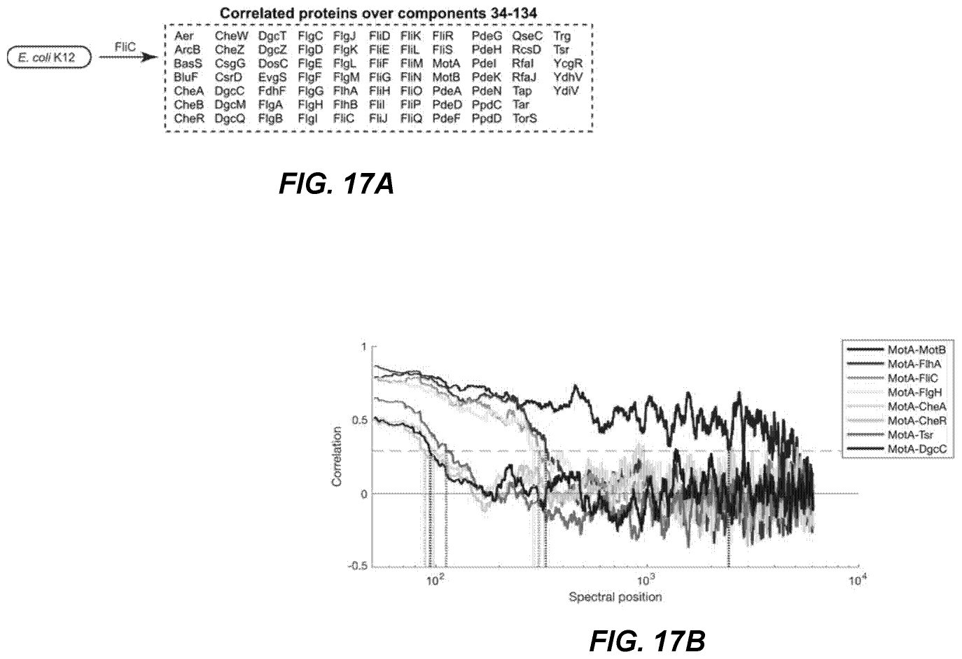

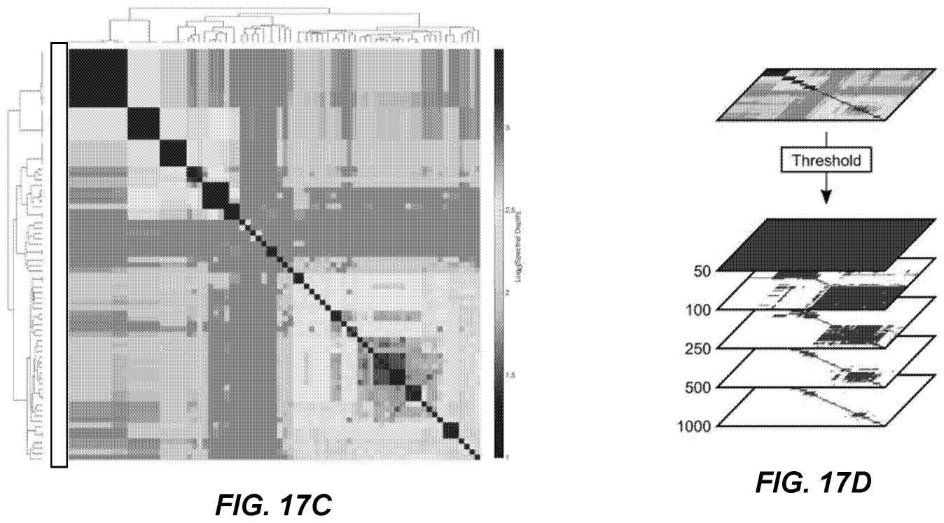

To develop a hierarchical model of the directed motility phenotype in E. coli K12 the inventors first identified all proteins (n=75) that were significantly correlated with FliC, the flagellar filament protein, over the first spectral window enriched for non-phylogenetic information (SVD34-SVD134) (FIG. 17A). For these proteins, the inventors computed pairwise spectral correlations across all 100 component windows of the SVD spectrum of D^{OGG}. The inventors observed that some pairs (e.g. MotA and MotB) remained significantly correlated as the spectral window was shifted deep into the SVD spectrum while other pairs (e.g. MotA and CheR) were significantly correlated only across the shallower regions of the SVD spectrum (FIG. 17B). The inventors computed the position at which the pairwise correlation first dropped below the significance threshold defined by the model of correlations between non-interacting proteins. The inventors define this position as the ‘spectral depth’ of correlation. The inventors computed the spectral depth for all pairs of proteins that were significantly correlated with FliC across SVD34 to SVD134 (FIG. 17C). Apply a threshold to spectral depth generates an adjacency matrix where a pixel value of ‘1’ indicates a pair of proteins that share a spectral depth that is as deep or deeper than the threshold value (FIG. 17D).

This adjacency matrix can be used to construct a protein interaction network at the thresholded spectral depth.

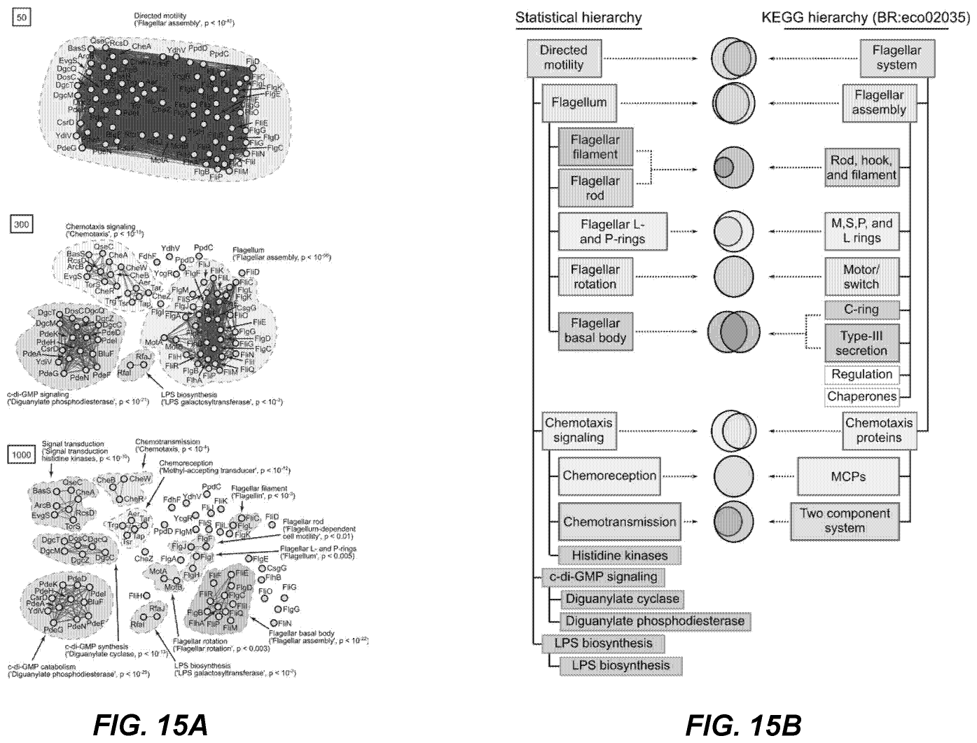

The inventors constructed protein interaction networks from the adjacency matrices produced by applying spectral depth thresholds of 50, 300, and 1000. At a spectral depth of 50, the inventors observed a single densely connected network devoid of obvious substructure (FIG. 15A, top panel). Gene set enrichment analysis (GSEA) indicated that this network was enriched for functional terms related to ‘flagellar system’ (p<10⁻⁴⁵) (Huang et al., 2009; Huang et al., 2009, Materials and Methods). Progressing to spectral depth of 300, the inventors observed that the network at 50 fractured into four discrete subnetworks (FIG. 15A, middle panel). These subnetworks were significantly enriched for terms related to ‘Chemotaxis signaling’ (p<10⁻¹⁵), ‘Flagellum’ (p<10⁻⁵⁶), ‘LPS biosynthesis’ (p<10⁻³), or ‘cyclic di-GMP signaling’ (p<10⁻²¹). Progressing to spectral depth of 1000, the subnetworks at 300 fractured further yielding 9 discrete subnetworks. Each subnetwork was significantly enriched for terms related to a specific function such as ‘cyclic di-GMP catabolism’ (p<10⁻²⁵) and ‘cyclic di-GMP synthesis’ (p<10⁻¹³) or ‘chemotransmission’ (p<10⁻⁴) and ‘chemoreception’ (p<10⁻¹²) (FIG. 15A, bottom panel).

Taken together the three network diagrams derived at spectral depths 50, 300, and 1000 depict a hierarchy of structure and function. Subnetworks observed at deeper spectral depths integrate to form the subnetworks observed at shallower spectral depths. As the subnetworks coalesced, the p-value associated with GSEA remained highly significant while the ontology of the significantly enriched terms changed. The inventors interpret these observations to mean that as the inventors ascend the statistical hierarchy, molecular descriptions of new biological functions emerge from the integration of functional units at lower levels.

The inventors compared the hierarchical model with the model of E. coli K12 motility detailed within the KEGG database (BR:eco02035) (Kanehisa et al., 2017) (FIG. 15B). The two models were similar in several ways. First, 44 of 55 of the proteins listed in the KEGG hierarchy also appeared in the statistical hierarchy. Second, 7 of the 12 categories listed in the KEGG hierarchy had a one-to-one correspondence with a subnetwork of the statistical model sharing an overlapping set of proteins and similar descriptive label. Finally, both hierarchies shared a conserved architecture consisting of the integration of chemoreception and chemotransmission into chemotaxis signaling, the integration of flagellar substructures into the flagellum, and at the most global level the integration of chemotaxis and the flagellum. The most striking difference was that the statistical hierarchy included subnetworks related to cyclic-di-GMP signaling and LPS biosynthesis which were absent from the KEGG hierarchy. Prior experimental studies have provided direct genetic evidence that these systems are involved in E. coli K12 motility (Paul et al., 2010, Walker et al., 2004).

Overall, of the 75 proteins in the hierarchical model of E. coli K12 motility, 44 (59%) were represented in the KEGG hierarchy, 28 (37%) were missing from the KEGG hierarchy but supported by prior experimental evidence in the literature, and only 3 (4%) remained unvalidated (CsgG, PpdD, TorS). Taken together, these results illustrate that identifying the E. coli K12 proteins that were significantly correlated with FliC and then serially thesholding their spectral depth produced a valid multiscale, hierarchical model of E. coli K12′s directed motility phenotype.

### Robustness and Generalizability of Defining Statistical Hierarchies Using Spectral Depth

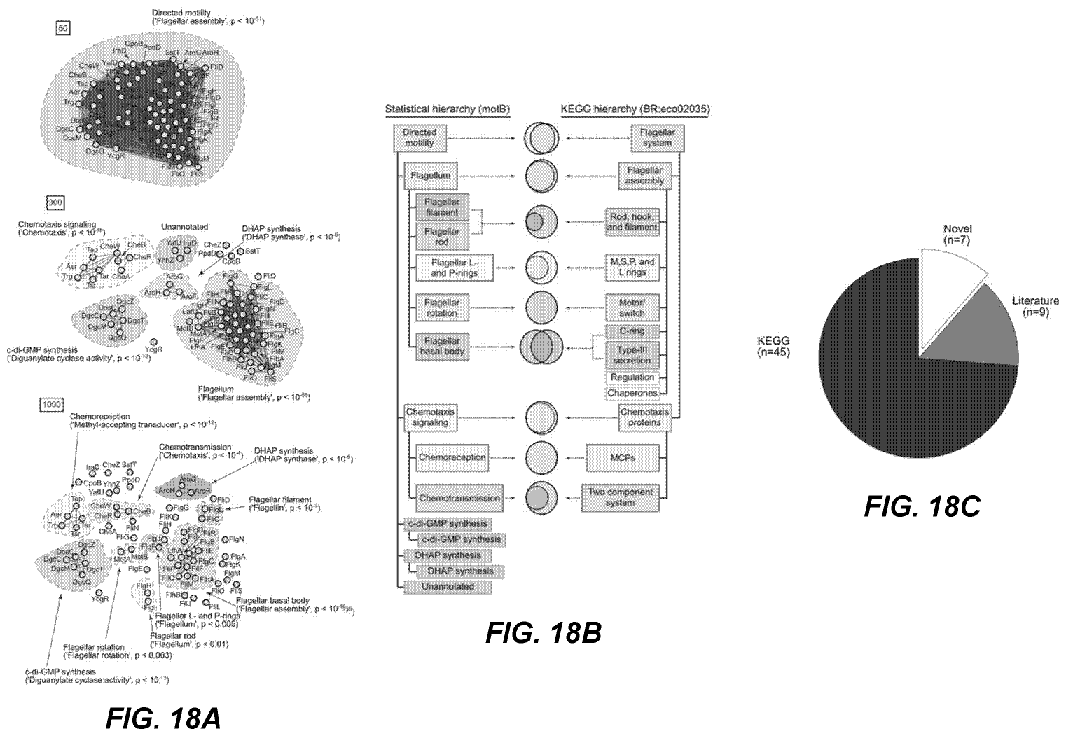

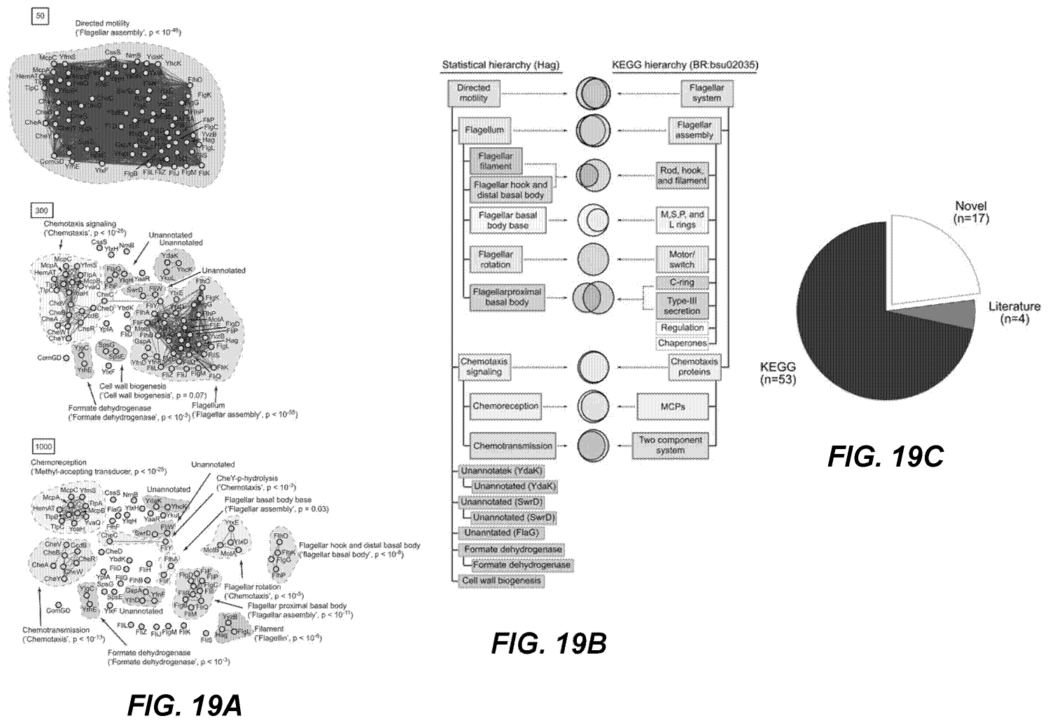

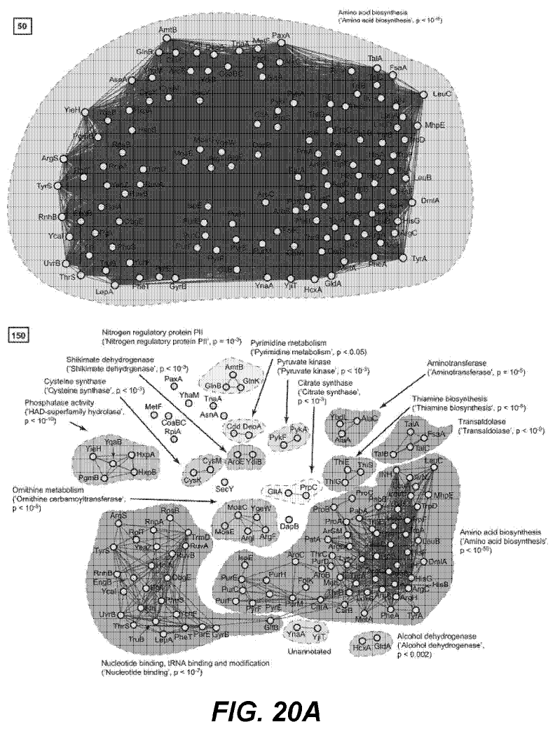

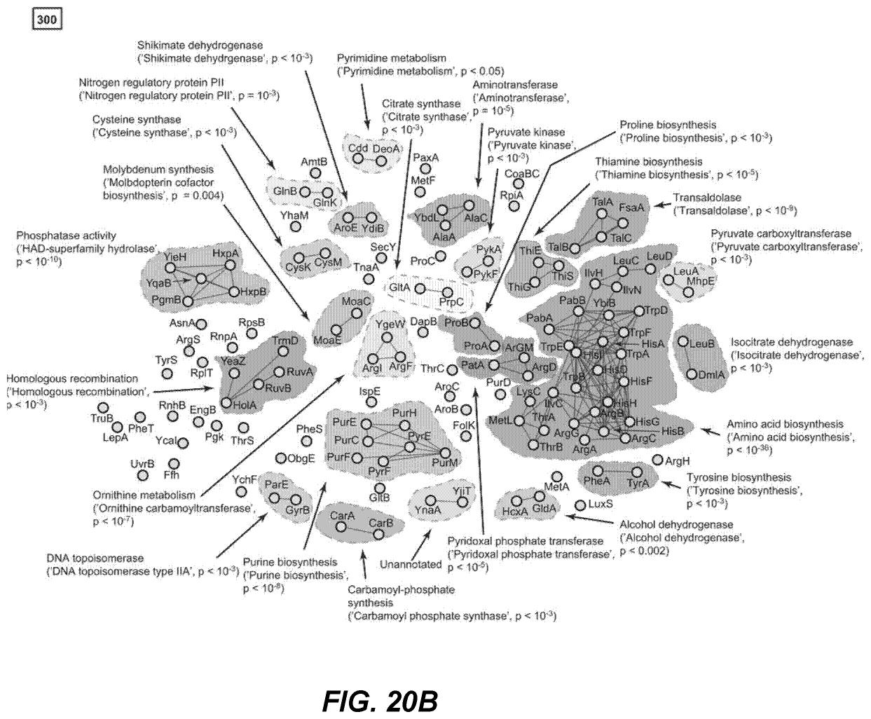

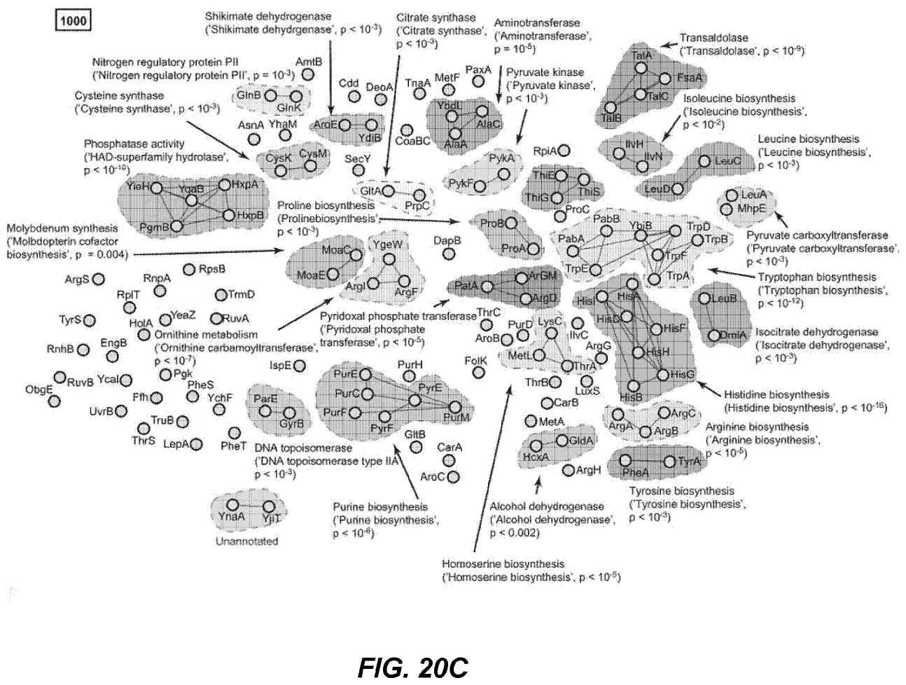

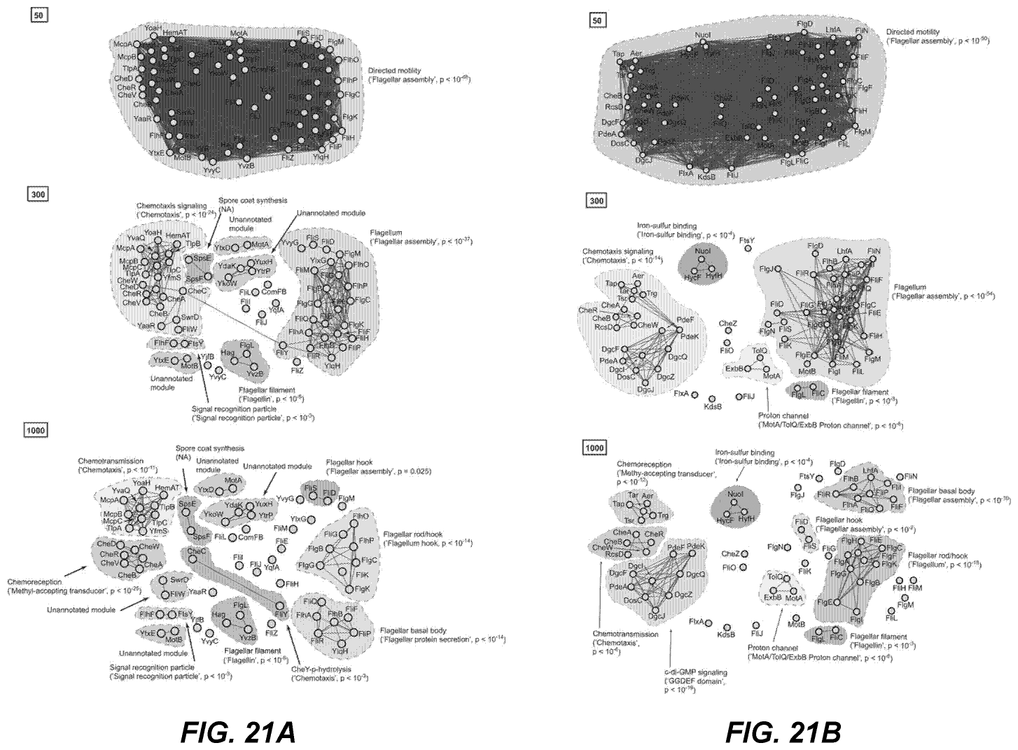

The inventors performed four additional to test the robustness and generalizability of the approach. First, the inventors characterized motility in E. coli K12 using MotB, the flagellar motor protein, as a query. The inventors found a similar hierarchical architecture as observed using FliC as the query with chemotaxis signaling, flagellum, and cyclic-di-GMP signaling modules appearing at spectral depth 300, and more fine-grained subnetworks appearing in deeper layers (FIG. 18). To test generalizability across organisms, the inventors created a model of motility in Bacillus subtilis 168 using its flagellar filament protein as a query (Hag). This analysis again produced a hierarchical model of motility that (i) recapitulated the corresponding KEGG hierarchy, (ii) identified proteins missing from the KEGG hierarchy that are known effectors of B. subtilis motility, and (iii) identified a small number of putative motility effectors (FIG. 19). Next, the inventors tested if the method could generalize to non-physically coupled pathways. The inventors produced a model of amino-acid metabolism in E. coli K12 using the query protein HisG, an enzyme involved in Histidine biosynthesis. The resultant hierarchical model identified 130 proteins that were densely connected at spectral depth 50. Progressing to deeper spectral depths revealed modules corresponding to specific functions, such as amino acid and nucleotide biosynthesis. At yet deeper spectral depths, modules enriched for proteins involved in the synthesis of specific amino acids became evident (FIG. 20). Finally, the inventors demonstrated that valid statistical models of B. subtilis 168 and E. coli K12 motility could be derived by serially thresholding spectral depth of correlations within the SVD spectrum of D^{domain} (FIG. 21). Taken all together, these analyses demonstrated that serially thresholding spectral depth produces a hierarchical model of biological pathways across different query proteins, organisms, pathways, and orthologous features.

### Using the Structure of a Statistically Defined Hierarchy to Aide in the Discovery of Novel Genotype-Phenotype Relationships

The hierarchical models produced by serially thresholding spectral depth recapitulated the known architecture of several well-studied biological phenotypes without incorporating any prior knowledge of biological organization. This motivated the hypothesis that these models could also reveal new biological organization that was not previously appreciated. The inventors tested this idea by generating a hierarchical model of motility in Pseudomonas aeruginosa, using it to assign both a general and specific function to a previously uncharacterized protein, and experimentally validating these predictions.

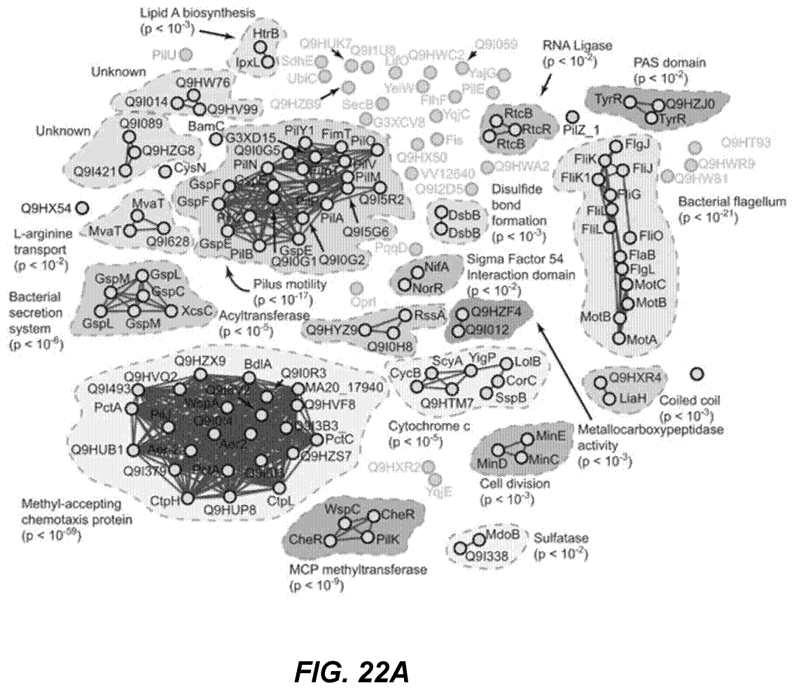

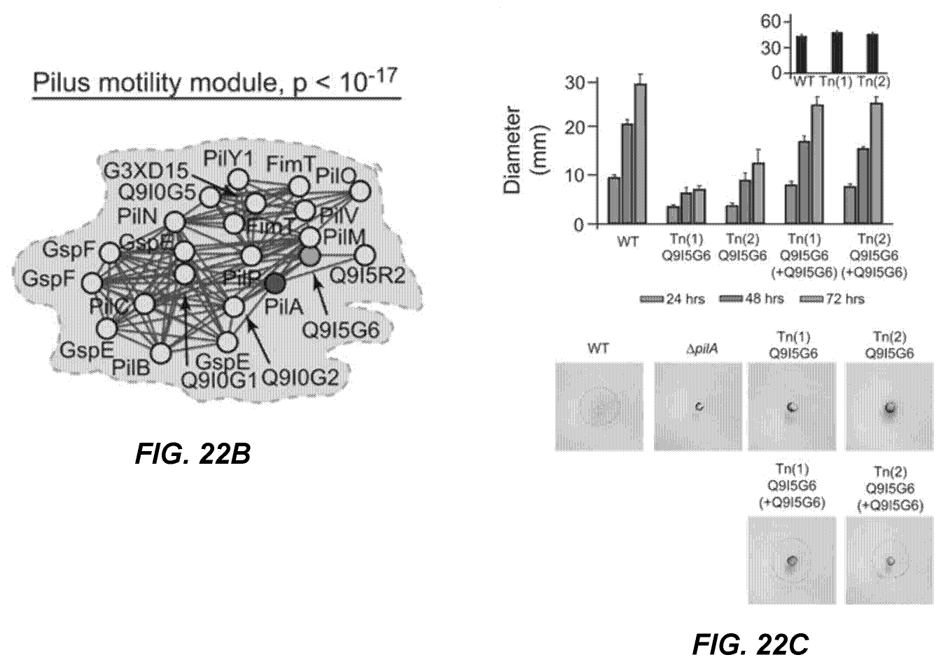

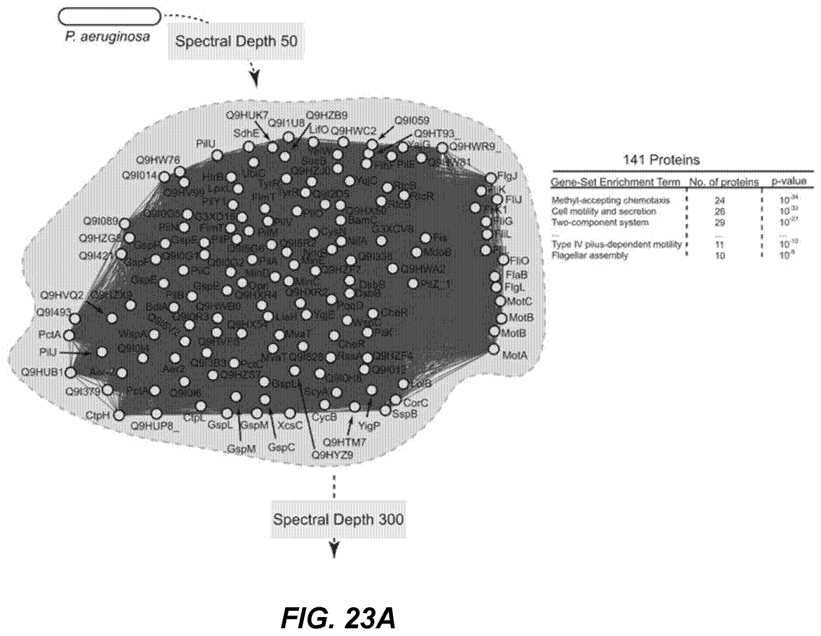

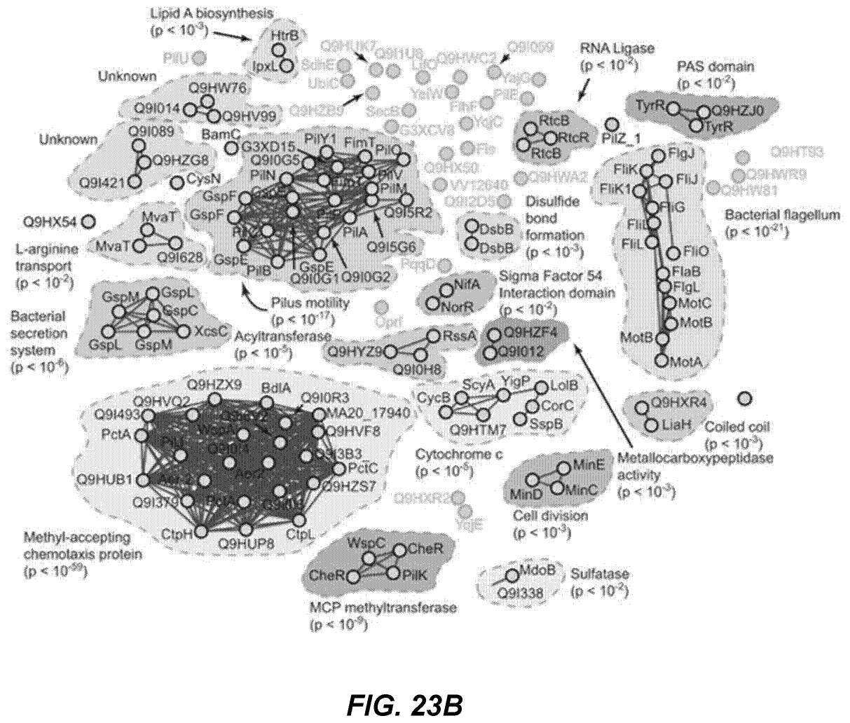

P. aeruginosa is uses two different types of motility—propulsive motility based on a flagellum and twitch motility based on a pilus (Kearns et. al., 2001; Rashid and Kornberg, 2001). Using PilA, a structural component of the pilus, as a query the inventors identified a network of 141 proteins as significantly correlated across SVD34 to SVD134. The inventors produced network configurations for these proteins using spectral depth thresholds of 50 and 300. At a spectral depth of 50 a single densely connected network was observed (FIG. 23A). Significantly enriched terms for this network were ‘methyl-accepting chemotaxis’ (p<10⁻³⁴), ‘cell motility and secretion’ (p<10⁻³³), ‘two-component system’ (p<10⁻²⁷), ‘type IV pilus-dependent motility’ (p<10⁻¹⁰), and ‘flagellar assembly’ (p<10⁻⁶), suggesting that these proteins are collectively involved in the global function of directed motility. At spectral depth 300, the network fractured into 18 different discrete subnetworks that were enriched for specific functions (FIG. 22A, FIG. 23B). The largest subnetworks were enriched for ‘methyl-accepting chemotaxis protein’ (p<10⁻⁵⁹), ‘pilus motility’ (p<10⁻¹⁷), or ‘bacterial flagellum’ (p<10⁻²¹). The inventors noted four proteins in the pilus subnetwork (Q915G6, Q915R2, Q910G2, Q910G1) that were annotated as ‘uncharacterized protein’ in UniProt (FIG. 22B). Further review of STRING, GO, BIOCYC, and PFAM revealed only that Q915G6 contains a ‘domain of unknown function’ (DUF4845). Based upon their membership in the pilus subnetwork at spectral depth 300, the inventors hypothesized that these proteins may contribute to the general function of directed motility by affecting the specific function of pilus-based motility. Furthermore, the lack of connections to the flagellum subnetwork suggested that these proteins would not impact flagellar based motility.

To test these predictions, the inventors screened single-gene transposon mutants of P. aeruginosa (PAO1) for twitch-based or flagellar-based motility using established experimental assays (Materials and Methods) (Kearns et. al., 2001; Rashid and Kornberg, 2001, Little et. al., 2018). Transposon mutants of Q915R2, Q910G2, and Q910G1 exhibited motility that was not significantly different from the parent strain in both assays. In contrast, the inventors found that two different transposon mutants of Q915G6 exhibited significantly reduced twitch motility velocity over 24, 48, and 72 hours compared to the parent strain (FIG. 22C, p<10⁻⁴ by Dunnett's multiple comparisons test). This phenotype resembled that of a knockout strain of pilA and was reversed upon trans-complementation. In contrast, flagellar-based motility of Q915G6 was not significantly different from that of the parent strain (FIG. 22C—inset, p>0.05). These results illustrate that Q915G6 is a previously unappreciated effector of the global directed motility phenotype in P. aeruginosa that specifically impacts twitch-based motility. As such, these experiments provide a proof of concept of how the hierarchical models may aid in discovering novel genotype-phenotype relationships.

### Discussion

Connecting genotype to phenotype is a central goal in biology. Achieving this goal requires understanding how the collection of proteins in a proteome interact at different scales spanning protein complexes, pathways, and cellular phenotypes. Here, the inventors have shown that a hierarchy of protein interaction networks can be extracted from analyzing covariation across an ensemble of bacterial proteomes. Key to this outcome were three important results. First, when the inventors spectrally decomposed proteome variation using SVD the inventors found that biological information mapped onto the SVD spectrum in a specific way: shallow components were enriched for phylogenetic relationships, deeper components for functional interactions between proteins in pathways, and even deeper components for physical interactions within protein complexes.

Second, the inventors found that spectral correlations measured across sets of SVD components defined features that informed accurate classification of protein pairs as non-interacting, indirect PPI, or direct PPI. Third, the inventors developed the concept of computing a spectral depth of correlation and found that serially thresholding spectral depth produced a hierarchical model of protein interaction networks. These models closely resembled the known hierarchical organization for several well-studied bacterial phenotypes. Finally, the inventors illustrated the utility of generating these unbiased hierarchical models by developing a model of motility in P. aeruginosa and using it to predict global and local functions for a previously uncharacterized protein.

### Discarding Global Components of Covariation Purifies PPI Information

Variation in bacterial proteomes arises from different sources of information and noise. An established approach to separating information from noise is to spectrally decompose the variation using SVD and then to identify which SVD components can explain more of the total variance than a random process (Wigner, 1967). This leads to considering the k most global SVD components (Franscescini et. al., 2016). In contrast, in the study, the inventors empirically mapped the distribution of biological information across the entire SVD spectrum. the results did not match the initial expectation that the most global components would inform the highest fidelity PPI predictions and minor components would solely contain noise. Instead, the inventors found that global components of covariation primarily reflected phylogeny, and PPI predictions based on these components were low quality. On the other hand, the inventors found that excluding global components while retaining minor components harboring a minuscule amount of variance produced high-fidelity PPI predictions. The inventors interpret these results to mean that percent variance per component does not indicate ‘importance’ of biological signal and discarding major components of covariation may actually purify functionally relevant information.

Spectral Depth: a Metric that Extracts a Hierarchy from the SVD Spectrum

SVD sequentially defines orthonormal vectors (components) that maximize the compression of the remaining unexplained data variance. The inventors found that there is a direct mapping between the position of a component within the SVD spectrum and level in the hierarchy of biological organization (FIG. 1C). Likely this mapping reflects intrinsic differences in the compressibility of biological variation arising from different hierarchical levels spanning protein complexes, pathways, and phenotypes. However, SVD alone does not reveal how the different levels in the hierarchy are related. Therefore, to extract a model of how protein interactions are hierarchically organized to generate a phenotype, the inventors devised the metric of spectral depth, the tracking of the persistence of correlations across the SVD spectrum. The inventors found that this metric enables predicting the integration of PPIs into complex structures approximating pathways and phenotypes.

### The Potential of Generalizing SCALES to Other Biological Systems

Re-application of these methods to a new dataset will require following the steps outlined herein: creating an ensemble, identifying relevant benchmarks, mapping the benchmarks onto SVD components, and developing a model of random spectral correlations to define spectral depth. SCALES represents a statistical way to describe emergence-the integration of individual components into layers of collective units of function. The property of emergence spans many biological systems, from proteins to ecosystems. Thus, SCALES may be a generally useful approach to learning the hierarchical architecture of biological systems.

### Materials and Methods

### Generating D^{OGG}

All bacterial proteomes (n=7,047) in the 2020_02 release of the Uniprot Reference Proteome database were downloaded on 05/20/2020 (FIG. 2) (The UniProt Consortium, 2019). OGGs were annotated using eggNOG-mapper V2 at the level of bacteria (‘@2’) (Huerta-Cepas et al., 2017; Huerta-Cepas et al., 2019). An OGG count matrix was assembled (D^{OGG}, FIG. 1A) where rows were defined as proteomes, columns were defined as OGGs, and the value in each cell was the number of annotations an OGG in a proteome. The number of annotations was used to preserve as much information as possible versus the strategy of considering binary occurrence. All OGGs present in fewer than 1% of the proteomes were removed leaving 10,177 unique columns in D^{OGG}.

### Singular Value Decomposition (SVD) of DOGG

Singular Value Decomposition (SVD) was performed on DOGG. First, the raw data matrix was centered and standardized by

$${z}_{ij} = \frac{( {d}_{ij} - \overline{{d}_{[ 1 , m ] , j}} )}{{sd}_{{d}_{[ 1 , m ] , j}}}$$

where zij is the ij^{th} element of the z-scored matrix Z^{OGG}, dij the ij^{th} element in the initial data matrix D^{OGG}, d̅_̅{̅[̅1̅,̅m̅]̅,̅j̅}̅ and sd_{d}_{_{[1,m],j}} are the mean and standard deviation of the j^{th} column vector of D^{OGG}, and m is the total number of rows in D^{OGG} (m=7,047). Z^{OGG} was factorized using SVD:

Z^{OGG}=U^{OGG}Σ^{OGG}V^{OGG}^{^{T}}

U^{OGG} is an m x K matrix where rows are proteomes, columns are the ‘left singular vectors’, and each element is the projection of a proteome onto a left singular vector. Σ^{OGG} is a m×n diagonal matrix where the K non-zero diagonal entries are ‘singular values’ and decrease in magnitude with position along the diagonal. V^{OGG} is a n×K matrix where the rows are OGGs, columns are the ‘right singular vectors’, and each element is the projection of an OGG onto a right singular vector. m and n are the number of rows (m=7,047) and columns (n=10,117) in Z^{OGG}. K is the total number of SVD components (K=7.047). The fraction of data variance explained by SVD component k is computed through the following equation:

$${var}^{k} = \frac{{\sum }_{kk}^{2}}{{\sum }_{i = 1}^{K} {\sum }_{ii}^{2}}$$

where var^{k} is the fractional variance explained by the k^{th} SVD component, Σkk and Σij are the k^{th} and i^{th} singular values respectively, and K is the total number of singular values. The plot of fractional variance per component versus component number are shown for D^{OGG} in FIG. 3D.

### Phylogeny Benchmarks

NCBI phylogenetic strings were mapped to the NCBI taxonometric IDs for each of the 7,047 bacteria represented in DOGG using taxonkit 5.0 (https://bioinf.shenwei.me/taxonkit/) on May 20, 2020. Five different benchmarks were generated corresponding to pairs of proteomes that share identical phylogenetic substrings down to the level of phylum (n=5,841,696), class (n=2,460,194), order (n=807,338), family (n=434,753), or genus (n=267,794).

### STRING Nonbinding Benchmark

STRING database annotations were downloaded for the Escherichia coli K12 proteome (STRING ID 511145) on Jul. 22, 2019. A benchmark was assembled to include all protein pairs (n=14,793) with a nonzero combined STRING score that did not share a ‘binding’ action annotation. This benchmark was expected to be enriched for indirect protein-protein interactions (PPIs).

### GO Terms Benchmark

Biological function' GO term annotations were mapped for the 4,391 proteins in the E. coli K12 proteome through the UniProtKB API. A benchmark was assembled containing the 79,794 protein pairs that share at least 1 GO term annotation. This benchmark likely contained a mixture of indirect and direct PPIs.

### STRING Benchmark

STRING database annotations were downloaded for the E. coli K12 proteome (STRING ID 511145) on Jul. 22, 2019. A benchmark was assembled comprised of all (n=20,216) protein pairs with a nonzero combined STRING score. This benchmark included a mixture of pairs with (n=14,793) and without (n=5,423) a ‘binding’ annotation and therefore is presumed to contain a mixture of direct and indirect PPIs.

### ECOCYC Benchmark

A previously published benchmark included 915 pairs of E. coli K12 proteins selected from the set of complexes in the ECOCYC database after intentionally excluding large complexes with greater than ten proteins to enrich for directly interacting pairs of proteins (Cong et al., 2019, Keseler et al., 2017). This benchmark is assumed to primarily represent direct PPIs.

### Coev+ Benchmark

A previously published set of 1,600 direct PPIs in E. coli K12 identified by a hybrid method combining the results of amino acid coevolution (AA Coev) and prior experimental data (Cong et al., 2019).

### PDB Benchmark

A previously published set of 809 direct PPIs in E. coli K12 selected by the criteria that they, or closely homologous proteins, have been observed to interact in a crystal structure in the PDB (Cong et al., 2019).

The type of biological information reflected in each benchmark is summarized in FIG. 1B.

### Computing Protein-Protein Spectral Correlations

A row vector in the matrix VOGG contains the projections of a single OGG onto each of the SVD components as described by:

[symbol]

_{i,[1,K]}=[fᵢ|1> . . . fᵢ|k> . . . fᵢ|K>]

where v_{i, [}i,k_{]} is the i^{th} row vector of the matrix V^{OGG}, fi is the OGG represented in row i of matrix V^{OGG}, fi_{|}k > is the projection of fl onto the SVD component k (1≤k≤K), and K is the total number of SVD components.

A vector representing the projections of a protein onto the SVD components was generated by averaging the vectors corresponding to the projections of all OGGs annotated within the protein:

$${Ω}_{l , [ 1 , K ]} = \frac{{\sum }_{f ∈ F} {v}_{{i}_{f} , [ 1 , K ]}}{\vert F \vert} = [ {Ω}_{l} \vert 1 > … {Ω}_{l} \vert k > … {Ω}_{l} \vert K > ]$$

where Ω/,_{[}a,b_{]} is a vector of the projections of protein l onto the set of SVD components ranging from component a to component b, 1≤a≤K−1, and 2≤b≤K.

The correlations between protein l and protein m within a spectral window was defined ρₗₘ^{a:b}=corr(Ω_{l,[a,b]},Ω_{m,[a,b]}) where ρₗₘ^{a:b} is the correlation between proteins l and m within the spectral window ranging from SVD component a to SVD component b, corr denotes the Pearson correlation, and Ω/,_{[}a,b_{]} and Ω_{[}a,b_{]} are the vectors containing the projections of proteins l and m onto the SVD components within the spectral window respectively. An example of this process is illustrated in FIG. 5G.

A model of random spectral correlations was generated by row shuffling the matrix V^{OGG} and then computing protein-protein spectral correlations as above (FIG. 6).

### Computing Mutual Information (MI) Between Spectral Correlations and Benchmarks of Biological Knowledge

For each phylogenetic benchmark, one-hundred bootstraps were generated consisting of equal numbers of randomly selected pairs of proteomes that do or do not share an identical phylogenetic substring annotation in the benchmark. For each protein interaction benchmark, one-thousand bootstraps were generated consisting of equal numbers of randomly selected pairs of proteins that do or do not share an interaction annotation in the benchmark. For bootstraps of both phylogenetic and protein interaction benchmarks, the number of pairs sharing an annotation was equal to the number of pairs indicated for each respective benchmark in the section.

‘Assembling Benchmarks that Collectively Represent the Hierarchy of Biological Organization’

Spectral correlations across all 5-component windows of the SVD spectrum between component 1 and component 3000 were computed for the proteome or protein pairs in each bootstrap. The uncertainty surrounding the spectral correlations within a single window was calculated as the differential entropy (Cover and Thomas, 2006):

$$H ( {ρ}^{a : b} ) = - {\sum }_{i} Δ p ( {ρ}_{i}^{a : b} ) {log}_{2} p ( {ρ}_{i}^{a : b} ) - {log}_{2} ( Δ )$$

where ρa:b is the vector of spectral correlations over the window ranging from components a to b for the pairs in the bootstrap, Hρa:bJ is the differential entropy of ρa:b, μa:b is a bin of correlation values produced by quantizing the continuous-valued correlations in ρa:b, p(pia:b) is the probability of observing a correlation value within ρa:b, and Δ is the width of the quantization bins. In the present study Δ=0.25.

Uncertainty surrounding spectral correlations given knowledge of the phylogenetic relationships or protein interactions annotated within a benchmark is described by the conditional entropy:

H(ρ^{a:b}|c0=p(c=1)H(ρ^{a:b}|c=1)+p(c=0)H(ρ^{a:b}|c=0)

where c is a binary vector that assumes a value of 1 or 0 if a pair of proteomes or proteins do or do not share an annotation in the corresponding benchmark respectively, Hρa:b|c is the uncertainty surrounding the windowed spectral correlations given knowledge of c, p(c=1) and p(c=0) are the probability of a 1 or 0 in c respectively, and Hρa:b|c=1) and Hρa:b|c=0) are the uncertainties surrounding the spectral correlations for subsets of pairs in the bootstrap that correspond to a value of 1 or 0 in c respectively and are computed using the differential entropy equation described above.

Finally, MI was computed as the difference between the uncertainty surrounding the spectral correlations with and without knowledge of the benchmark:

I(ρ^{a:b}, c)=H(ρ^{a:b})−H(ρ^{a:b}|c) where Iρa:b, cJ is the estimate for the MI shared between the spectral correlations and the benchmark. A model of random MI was generated by computing the MI shared between the spectral correlations within row-shuffled versions of U^{OGG} or V^{OGG} and the benchmarks of phylogeny and protein interactions respectively (FIG. 6). The distributions of MI estimates for the different benchmarks arising from the data or random model are summarized in FIG. 7.
Calculation of MI Cumulative Distribution Functions (cdfs) shown in FIG. 1C

Each point in the MI cdfs shown in FIG. 1C was computed as (for the window centered on component w of the SVD)

$${cdf}_{w} = \frac{{\sum }_{i = 1}^{w} ( {I}_{i}^{data} - {I}_{i}^{random} )}{{\sum }_{i = 1}^{W} ( {I}_{i}^{data} - {I}_{i}^{random} )}$$

where cdfw is the value of the cdf at spectral position w, Ii^{data} is the MI observed in window i, Ii^{random} is the MI produced by random correlations in window i, W is the total number of windows, and 1≤w≤W. Because the inventors considered 5-component spectral windows within the first 3000 components, W=2997.
Training and Validating Random Forests (RF) Models for Predicting PPIs in E. coli K12 Using MIWSC's

### Assembling a ‘Gold-Standard’ Dataset

A ‘gold-standard’ dataset for E. coli K12 PPIs was assembled and consisted of 72,000 not-interacting, 1,226 indirect PPIs, and 72 direct PPIs. All pairs defined as ‘direct PPI’ satisfied three criteria: they shared amino-acid level coevolution (Coev+ benchmark), were annotated in the same protein complex in the ECOCYC benchmark, and interacted in the PDB benchmark. All indirect PPIs were selected based on the following criteria: they shared a ‘non-binding’ type interaction annotation in the STRING Nonbinding benchmark, shared a ‘biological function’ interaction in the GO benchmark, and did not share an interaction annotation in any of the benchmarks of direct PPIs (Coev+, ECOCYC, or PDB). The ‘not-interacting’ pairs did not share an interaction annotation in any of the benchmarks (GO, STRING Nonbinding, STRING, Coev+, ECOCYC, or PDB). The not-interacting set was subsampled to exceed the number of physically interacting pairs by 1000-fold (Rajagopala et al., 2014; Cong et al., 2019).

The gold standard pairs were randomly partitioned into training (60%) and validation (40%) datasets. Fifty such random partitions were generated to assess the reproducibility of the results of the machine-learning task described below.

### Training RF Models

RF models consisting of 100 decision trees were trained to classify pairs of proteins in E. coli K12 as not-interacting, indirect PPIs, or direct PPIs by feeding the labeled training set examples to the TreeBagger algorithm (Matlab, v2020a). This process was repeated for each random partition of the gold-standard dataset yielding an ensemble of 50 RF models per feature.

### Validating RF Models Using the Validation Dataset

Each trained RF model was subjected to three validation tasks of classifying interaction types for unlabeled pairs of E. coli K12 proteins in the validation portion of the gold-standard (40%) (FIG. 2—figure supplement 1, FIG. 2—figure supplement 2A). The model performance was evaluated by computing an F-score for each interaction type (not-interacting, indirect PPIs, direct PPIs), where F-score is the harmonic mean of precision and recall, precision is the ratio of the number of correctly predicted interactions within a class to the total number of predicted interactions in a class, and recall is the ratio of the number of correctly predicted interactions within a class to the total number of interactions of that class.

### Training and Validating RF Models on Quantitative Features of Existing Methods

For each feature extracted from existing methods described below, RF models were trained and validated using the identical protocol as for MIWSCs (described in the section Training and validating Random Forests (RF) models for predicting PPIs in E. coli K12 using MIWSC's).

### Existing Experimental Features

Previously published datasets derived from large scale experimental PPI screens in E. coli K12 were used to generate a set of four different experimental features including: gene interaction scores from a gene epistasis screen (Epistasis, n=41,820), sum log-likelihood scores from an affinity purification mass spectrometry screen (APMS1, n=12,801), protein interaction scores from an affinity purification mass spectrometry screen (APMS2, n=291), and binary pairs from a yeast-two hybrid screen (Y2H, n=1,766) (Rajagopala et al., 2014; Babu et al., 2014; Babu et al., 2018; Hu et al., 2009).

### Existing Computational Features

Gene co-occurrence, gene fusion, and gene neighborhood subscores for E. coli K12 (STRING ID 511145) were extracted from the STRING database on Jul. 22, 2019 (Szklarczyk et al., 2019; Rajagopala et al., 2014; Babu et al., 2014; Babu et al., 2018; Hu et al., 2009). Any pairs without an interaction annotation in the STRING database were assigned a subscore of zero.

Binary MI (b-MI) Feature

The b-MI feature was modeled after the popular phylogenetic profiling method of Pelligrini and colleagues (Pellegrini et al., 1999). First, a binary OGG content matrix was definedas follows:

$${B}^{OGG} = \{ \begin{matrix}1 , & {D}^{OGG} > 0. \\ 0 , & otherwise\end{matrix} .$$

Where B^{OGG} is the binary OGG content matrix and has the same dimensions as D^{OGG}.

The phylogenetic profile of an OGG was defined as: ppⱼ=B^{OGG}_{[1,m],j} where ppj is the phylogenetic profile of OGGj, and BOGG_{[1,m],j} and m are the j^{th} column vector and number of rows in the B^{OGG} respectively. For each pair of proteins in the proteome of E. coli K12, a phylogenetic profiling feature of protein coevolution was defined as the average of the MI shared between the profiles of all pairs of OGGs encoded in the two proteins:

$$b - {MI}_{lp} = \frac{{\sum }_{j ∈ J} {\sum }_{k ∈ K} I ( {pp}_{j} , {pp}_{k} )}{\vert J \vert * \vert K \vert}$$

Where b-MIl,p is the MI shared between the phylogenetic profiles of protein l and protein p, J and K are the sets of OGGs encoded in proteins l and p respectively, j and k are elements of J and K respectively, ppj and ppk are the phylogenetic profiles of j and k respectively, Ippj, ppk) is the MI shared between ppj and ppk computed using Shannon's classic formulation for the MI between two discrete random variables (Shannon, 1970; Cover and Thomas, 2006), and |J| and |K| are the number of elements in J and K respectively.

### Covariation Feature

The covariation between a pair of OGGs was described by:

$${Cov}_{jk} = \frac{1}{m} {\sum }_{i = 1}^{m} ( {d}_{ij} - \overline{{d}_{i , [ 1 , n ]}} ) ( {d}_{ik} - \overline{{d}_{i , [ 1 , n ]}} )$$

where Covjk is the covariation between OGGs j and k, m and n are the number of proteomes (rows) and OGGs (columns) in D^{OGG} respectively, dij and dik are the number of annotations of OGGs j and k in proteome i respectively, and d̅_̅{̅l̅,̅[̅1̅,̅n̅]̅}̅ is the average number of OGG annotations in proteome i obtained by averaging the corresponding row vector in D^{OGG}. For each pair of proteins in the proteome of E. coli K12, protein covariation was defined as the average of the covariation shared between all pairs of OGGs encoded in the two proteins:

$${Cov}_{lp}^{protein} = \frac{{\sum }_{j ∈ J} {\sum }_{k ∈ K} {Cov}_{jk}}{\vert J \vert * \vert K \vert}$$

where Co [symbol] ₗₚ^{protein} is the covariation feature of interaction between protein l and protein p, J and K are the sets OGGs encoded in proteins l and p respectively, j and k are elements of J and K respectively, Covjk is the covariation between OGGs j and k, and |J| and |K| are the number of elements in J and K respectively.

### PCA-Based Spectral Correlations Features

These features were inspired by the approach of Franceschini and colleagues and the typical use of SVD to produce a low rank approximation of the initial data matrix in an effort to ‘denoise’ the data (Franceschini et al., 2016). For each pair of proteins in the E. coli K12 proteome spectral correlations were computed as described in the section ‘Computing protein-protein spectral correlations’ over windows ranging from component 1 to component k, where k=5, 10, 20, 50, 100, 500, 1000, 5000, or 7047.

### Validating RF Models in Two Additional Validation Tasks

### Training Dataset Task

Each decision tree within an RF model was tasked with predicting interaction classes for the out-of-bag examples from the training datasets. F-scores were computed for the consensus predictions of each model.

### Comprehensive Benchmark Task

Biological interactions are typically sparse: the number of not-interacting pairs of proteins vastly outnumber the number of interacting pairs. As such, the inventors desired to challenge each of the RF models in a validation task reflective of this asymmetry. To do so, each RF model was tasked with predicting classes for all pairs of proteins in the E. coli K12 proteome after exclusion of pairs used in the gold-standard dataset. These predictions were validated against four different comprehensive benchmarks: the indirect PPIs in the STRING Nonbinding benchmark (n=5,423 indirect PPIs, 9,637,213 not-interacting), the mixed indirect/direct PPIs in the GO (n=79,794 indirect or direct PPIs, 9,562,842 not-interacting) and STRING benchmarks (n=20,216 indirect or direct PPIs, 9,622,420 not-interacting), and the direct PPIs in the entire PDB benchmark (n=809 direct PPIs, 9,614,827 not-interacting).

### Predicting Proteome-Wide Direct PPIs for 11 Phylogenetically Unrelated Bacteria

### Proteomes Represented in D^{OGG}

Each of the fifty RF models trained to classify interactions in E. coli K12 using MIWSCs were used to predict proteome-wide indirect and direct PPIs in the following bacteria (Uniprot Proteome ID, NCBI taxonomy ID in parentheses): Aliivibrio fischeri ES114 (UP000000537, 312309), Azotobacter vinelandii DJ (UP000002424, 322710), Bacillus subtilis 168 (UP000001570, 224308), Caulobacter vibrioides (UP000053705, 155892), Helicobacter pylori 26695 (UP000000429, 85962), Mycobacterium tuberculosis H37Rv (UP000001584, 83332), Mycoplasma genitalium G37 (UP000000807, 243273), Pseudomonas fluorescens F113 (UP000005437, 1114970), Staphylococcus aureus NCTC 8325 (UP000008816, 93061), Streptomyces coelicolor A3(2) (UP000001973, 100226), Synechocystis sp. PCC 6803 (UP000001425, 1111708). For each proteome, a set of consensus PPIs was defined as those for which a majority of the models (>25) produced the same classification of ‘indirect PPI’ or ‘direct PPI’.

### Proteomes not Represented in D^{OGG}

To predict interactions for a proteome that was not represented in D^{OGG} (ex. Azotobacter vinelandii DJ, UP000002424, 322710), OGGs were mapped using EggNOG mapper V2 and MIWSCs were extracted using the OGG projections in V^{OGG} (Huerta-Cepas, 2017; Huerta-Cepas, 2019). These features were used to predict proteome-wide indirect and direct PPIs as described for the Uniprot Reference Proteomes above.

### Validating Direct PPI Predictions Against Experimental Evidence in the STRING Database

The predicted direct PPIs were benchmarked against the sets of interactions in the STRING database with a non-zero experimental subchannel score for E. coli K12 and the eleven additional organisms described above.

Relating the Structure of Protein Covariation Defined by Domain-Based Features with the Hierarchy of Biological Organization as Shown in FIG. 12C

### Computing Domain-Based Proteome-Proteome and Protein-Protein Spectral Corelations

Domain-based proteome-proteome and protein-protein spectral correlations were computed in an identical fashion as described for OGG-based spectral correlations as described in the sections ‘Computing proteome-proteome spectral correlations’ and ‘Computing protein-protein spectral correlations’ respectively.

### Computing MI Shared Between Domain-Based Spectral Correlations and Benchmarks of Biological Organization

The MI shared between spectral correlations within all 5-component windows of the SVD spectrum of D^{domain}, ranging from component 1 to component 3000, and benchmarks of biological organization was computed in the identical way and for the identical benchmarks as described in the section Computing Mutual Information (MI) between spectral correlations and benchmarks of biological knowledge and is shown and compared to a random model in FIG. 13.

Calculation of Domain-Based MI cdfs Shown in FIG. 12C

MI cdfs shown in FIG. 12C were computed in an identical fashion as described in the section ‘Calculation of MI cumulative distribution functions (cdfs) shown in FIG. 1C.

### Training and Validating MIWSC Domain-Based RF Models to Infer PPIs

RF models were trained using MIWSCdomains, validated, and compared to existing methods according to the same protocols described in the sections Training and validating Random Forests (RF) models for predicting PPIs in E. coli K12 using MIWSC's, Training and validating

RF models on quantitative features of existing methods, and Validating RF models in two additional validation tasks.

Gene-set enrichment analysis performed on statistical model of E. coli K12 motility

Gene-set enrichment analysis (GSEA) was performed on the sets of proteins defined by the statistical modules using DAVID analysis (v6.8). The ontological term with the lowest p-value is indicated for each statistical module shown in FIG. 15.

### Evaluating the Robustness and Generalizability of Predicting the Hierarchical Organization of Biological Pathways Using Spectral Correlations

For the additional analyses characterizing motility in E. coli K12 using MotB, characterizing motility in B. subtilis using Hag, characterizing amino acid metabolism in E. coli K12 using HisG, and characterizing motility in E. coli K12 and B. subtilis using FliC and Hag respectively with domain-based spectral correlations, the network graphs shown in FIGS. 18-21 were generated following the identical approach described in the section ‘Predicting the hierarchical organization of the motility pathway in E. coli K12’. Note that for the domain-based characterization, a separate null model for spectral correlations in D^{domain} was developed and applied as described in the section Developing a null model of random protein-protein spectral correlations within the SVD spectrum of D^{OGG}. Gene-set enrichment analysis was performed for each example of a statistically derived pathway in an identical fashion as described in the section Gene-set enrichment analysis performed on statistical model of E. coli K12 motility.

Assaying Strains of P. aeruginosa for Pilus and Flagellar Motility

All P. aeruginosa strains used in this study were ordered from the Manoil Lab. All strains were grown at 37° C. on LB supplemented with 25 μg/ml irgasan and gentamicin (75 μg/ml) as necessary. E. coli XL1-Blue was maintained on LB agar plates with gentamicin (15 μg/ml) as necessary.

P. aeruginosa transposon mutants of interest were ordered from the Manoil Lab. P. aeruginosa growth was at 37° C. on LB supplemented with 25 μg/ml irgasan and gentamicin (75 μg/ml) as necessary. Strains were assayed for subsurface twitching motility as previously described (Alm and Mattick, 1995; Little et al, 2018). Strains were grown overnight and stab inoculated in the interstitial space between the basal surface of 1.0% LB agar and a plastic petri dish. Plates were incubated for 48 hours at 37° C. Agar was removed and cells attached to the plate were stained with 0.5% crystal violet; twitch zone diameter was measured and plates were imaged.

Surface twitching motility assays were performed as previously described (Little et al., 2018; Kearns et al., 2001). P. aeruginosa strains of interest were grown overnight and concentrated in morpholinepropanesulfonic acid (MOPS) buffer (10 mM MOPS, 8 mM MgSO4, pH 7.6). A 2.5 μl volume of the MOPS buffered bacterial suspension was spotted onto buffered twitching motility plates (10 mM Tris, 8 mM MgSO4, 1 mM NaPO4, 0.5% glucose, 1.5% agar, pH 7.6) and was incubated for 24 hours at 37° C. The twitching zone was measured and imaged.

Swimming motility was performed as previously described (Rashid and Kornberg, 2000).

Overnight cultures were stab inoculated into the surface of LB-0.3% bacto agar and were incubated for 24 hours at 37° C. The resulting swimming zone was measured.

For complementation of genes of interest into P. aeruginosa strains, the complementation vector pBBR1-MCS5-PA0769 was created using Gibson assembly. The vector was transferred to P. aeruginosa by electroporation using 2.2 kV in a 2 mm gap cuvette, and subsequent selection using gentamicin.

All of the methods disclosed and claimed herein can be made and executed without undue experimentation in light of the present disclosure. While the compositions and methods of this invention have been described in terms of specific embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

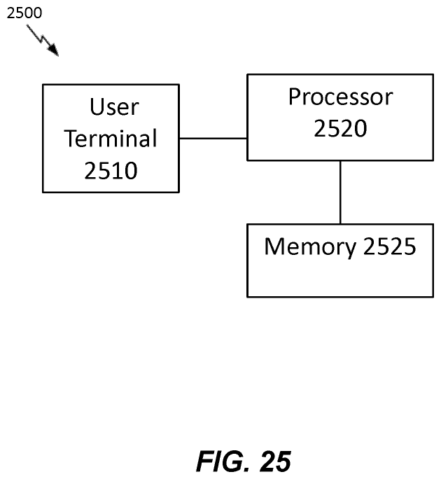

The techniques disclosed herein may be implemented in a system, such as system 255 illustrated in FIG. 25, configured with capabilities and functionality for analyzing higher-order biological interactions in accordance with embodiments of the present disclosure. In aspects, system 2500 may include input/output (I/O) module 2510, display 2515, processor 2520, and memory 2525. These components, and their individual components, may cooperatively operate to provide functionality in accordance with the discussion herein.

It is noted that the functional blocks, and components thereof, of system 2500 of embodiments may be implemented using processors, electronic devices, hardware devices, electronic components, logical circuits, memories, software codes, firmware codes, etc., or any combination thereof. For example, one or more functional blocks, or some portion thereof, may be implemented as discrete gate or transistor logic, discrete hardware components, or combinations thereof configured to provide logic for performing the functions described herein. Additionally or alternatively, when implemented in software, one or more of the functional blocks, or some portion thereof, may comprise code segments operable upon a processor to provide logic for performing the functions described herein.

It is also noted that various components of system 2500 are illustrated as single and separate components. However, it will be appreciated that each of the various illustrated components may be implemented as a single component (e.g., a single application, server module, etc.), may be functional components of a single component, or the functionality of these various components may be distributed over multiple devices/components. In such aspects, the functionality of each respective component may be aggregated from the functionality of multiple modules residing in a single, or in multiple devices.

It is further noted that functionalities described with reference to each of the different functional blocks of system 2500 described herein is provided for purposes of illustration, rather than by way of limitation and that functionalities described as being provided by different functional blocks may be combined into a single component or may be provided via computing resources, such as distributed resources disposed in a cloud-based environment accessible over a network.

Processor 2520 may comprise a processor, a microprocessor, a controller, a microcontroller, a plurality of microprocessors, an application-specific integrated circuit (ASIC), an application-specific standard product (ASSP), or any combination thereof, and may be configured to execute instructions to perform operations in accordance with the disclosure herein.

In some aspects, as noted above, implementations of processor 2520 may comprise code segments (e.g., software, firmware, and/or hardware logic) executable in hardware, such as a processor, to perform the tasks and functions described herein. In yet other aspects, processor 2520 may be implemented as a combination of hardware and software. Processor 2520 may be communicatively coupled to memory 2525.

Memory 2525 may comprise one or more semiconductor memory devices, read only memory (ROM) devices, random access memory (RAM) devices, one or more hard disk drives (HDDs), flash memory devices, solid state drives (SSDs), erasable ROM (EROM), compact disk ROM (CD-ROM), optical disks, other devices configured to store data in a persistent or non-persistent state, network memory, cloud memory, local memory, or a combination of different memory devices. Memory 2525 may comprise a processor readable medium configured to store one or more instruction sets (e.g., software, firmware, etc.) which, when executed by a processor (e.g., one or more processors of processor 2520), perform tasks and functions as described herein.

System 2500 may include user terminal 2510. User terminal 2510 may be implemented as a mobile device, a smartphone, a tablet computing device, a personal computing device, a laptop computing device, a desktop computing device, a computer system of a vehicle, a personal digital assistant (PDA), a smart watch, another type of wired and/or wireless computing device, or any part thereof. User terminal 2510 may be configured to facilitate input and output operations in accordance with aspects of the present disclosure, such as by generating and/or displaying a graphical user interface (GUI).

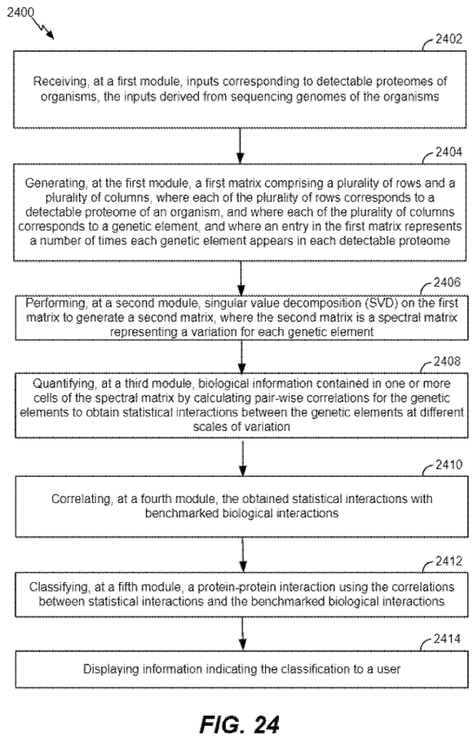

FIG. 24 shows a high level flow diagram 2400 of operation of a system configured in accordance with embodiments of the present disclosure for analyzing higher-order biological interactions. In some implementations, the operations of the method 2400 may be stored as instructions that, when executed by one or more processors, cause the one or more processors to perform the operations of the method 2400. The operations of the method 2400 may be implemented on a system, such as system 2500 of FIG. 25.

The method 2400 includes receiving, at a first module, inputs corresponding to detectable proteomes of organisms , the inputs derived from sequencing genomes of the organisms at 2402. The method 2400 includes generating, at the first module (e.g., a first module stored in memory 2525 and executed by processor 2520), a first matrix comprising a plurality of rows and a plurality of columns at 2404, where each of the plurality of rows corresponds to a detectable proteome of an organism, and where each of the plurality of columns corresponds to a genetic element. In some aspects, an entry in the first matrix represents a number of times each genetic element appears in each detectable proteome, where a genetic element may include, for example, an orthologous gene group, a conserved protein domain, etc.

The method 2400 includes performing, at a second module (e.g., a second module stored in memory 2525 and executed by processor 2520), singular value decomposition (SVD) on the first matrix to generate a second matrix at 2406, where the second matrix is a spectral matrix representing a variation for each genetic element. The method 2400 includes quantifying, at a third module (e.g., a third module stored in memory 2525 and executed by processor 2520), biological information contained in one or more cells of the spectral matrix by calculating pair-wise correlations for the genetic elements to obtain statistical interactions between the genetic elements at different scales of variation at 2408.

The method 2400 includes correlating, at a fourth module (e.g., a fourth module stored in memory 2525 and executed by processor 2520), the obtained statistical interactions with benchmarked biological interactions at 2410. The method 2400 includes classifying, at a fifth module (e.g., a fifth module stored in memory 2525 and executed by processor 2520), a protein-protein interaction using the correlations between statistical interactions and the benchmarked biological interactions at 2412. The method includes displaying (e.g., at user terminal 2510) information indicating the classification to a user at 2414.

Although aspects of the present disclosure and their advantages have been described in detail, it should be understood that various changes, substitutions and alterations can be made herein without departing from the scope of the disclosure as defined by the appended claims. Moreover, the scope of the present application is not intended to be limited to the particular implementations of the process, machine, manufacture, composition of matter, means, methods and steps described in the specification. As one of ordinary skill in the art will readily appreciate from the present disclosure, processes, machines, manufacture, compositions of matter, means, methods, or steps, presently existing or later to be developed that perform substantially the same function or achieve substantially the same result as the corresponding implementations described herein may be utilized according to the present disclosure. Accordingly, the appended claims are intended to include within their scope such processes, machines, manufacture, compositions of matter, means, methods, or steps. Moreover, the scope of the present application is not intended to be limited to the particular implementations of the process, machine, manufacture, composition of matter, means, methods and steps described in the specification.

### REFERENCES

The following references, and those cited elsewhere herein, to the extent that they provide exemplary procedural or other details supplementary to those set forth herein, are specifically incorporated herein by reference.

1. Barabasi AL, Zoltan O, (2004) Network Biology: Understanding the cell's functional organization Nat. Rev. Gen. 5:101-113. doi.org/10.1038/nrg1272

2. Chuang HY, Hofree M, Ideker TA, (2010) A decade of systems biology. Annu. Rev. Cell Dev. Biol. 26:721-744. doi.org/10.1146/annurev-cellbio-100109-104122

3. Hartwell LH, Hopfield JJ, Leibler S, Murray AW, (1999) From molecular to modular cell biology. Nature 402:C47-C52. doi.org/10.1038/3501154

4. Costanzo M. et al., (2016) A global genetic interaction network maps a wiring diagram of cellular function. Science 353:aaf1420. doi.org/10.1126/science.aaf1420

5. Papin J, Reed J, Paulsson BO, (2004) Hierarchical thinking in network biology: the unbiased modularization of biochemical networks. Trends in Biochemical Sciences 29:641-647. doi.org/10.1016/j.tibs.2004.10.001

6. Ravasz E, (2009) Detecting Hierarchical Modularity in Biological Networks. Methods Mol Biol. 541:145-60. doi.org/10.1007/978-1-59745-243-4_7

7. Nurse P, (2008) Life, logic and information. Nature 454:424-426. doi.org/10.1038/454424a

8. Rajagopala S et al., (2014) The binary protein-protein interaction landscape of Escherichia coli. Nat. Biotechnol. 32:285-290. doi.org/10.1038/nbt.2831

9. Scheonrock A et al., (2017) Evolution of protein-protein interaction networks in yeast. PLoS One 12:e0171920. doi.org/10.1371/journal.pone.0171920

10. Hauser R et al., (2014) A second-generation protein-protein interaction network of Helicobacter pylori. Mol. Cell. Proteomics 13:1318-1329. doi.org/10.1074/mcp.O113.033571

11. Koo B et al., (2017) Construction and analysis of two genome-scale deletion libraries for Bacillus subtilis. Cell Syst. 4:291-305.e.7. doi.org/10.1016/j.cels.2016.12.013

12. Luck K et al., (2020) A reference map of the human binary protein interactome. Nature 580:402-408. doi.org/10.1038/s41586-020-2188-x

13. Eisen J, (1998) Phylgenomics: Improving functional predictions for uncharacterized genes by evolutionary analysis. Genome Research 8:163-167. doi.org/10.1101/gr.8.3.163

14. Pellegrini M et al., (1999) Assigning protein functions by comparative genome analysis: Protein phylogenetic profiles. Proc. Natl. Acad. Sci. 96:4285-4288. doi.org/10.1073/pnas.96.8.4285

15. Enright A, Iliopoulos I, Kyrpides N, Ouzounis C, (1999) Protein interaction maps for complete genomes based on gene fusion events. Nature 402:86-90. doi.org/10.1038/47056

16. Valencia A, Pazos F, (2002) Computational methods for the prediction of protein interactions. Curr. Opinion in Struct. Biol. 12: 368-373. doi.org/10.1016/s0959-440×(02)00333-0

17. Croce G, Gueudre T, Cuevas M, Keidel V, Figliuzzi M, Szurmant H, Weigt M, (2019) A mult-scale coevolutionary approach to predict interactions between protein domains. PLOS Comput. Biol. 15:e1006891. doi.org/10.1371/journal.pcbi. 1006891

18. Cong Q, Anishchenko I, Ovchinnikov S, Baker D, (2019) Protein interaction networks revealed by proteome coevolution. Science 365:185-189. doi.org/10.1126/science.aaw6718

19. Green A, Elhabashy H, Brock K, Maddamsetti R, Kohlbacher O, Marks D, (2021) Large-scale discovery of protein interactions at residue resolution using co-evolution calculated from genomic sequences. Nat. Commun. 12:1396. doi.org/10.1093/bioinformatics/bty862

20. Szklarczyk D et al., (2018) STRING v11: protein-protein association networks with increased coverage, supporting functional discovery in genome-wide experimental datasets. Nucleic Acids Res. 47:D607-D613. doi.org/10.1093/nar/gkyl131

21. Kuzmin E et al., (2018) Systematic analysis of complex genetic interactions. Science 360:eaao1729. doi.org/10.1126/science.aao1729

22. Kanehisa M, Goto S, (2000) KEGG: Kyoto Encyclopedia of Genes and Genomes. Nucleic Acids Res. 28:27-30. doi.org/10.1093/nar/28.1.27

23. Kanehisa M, (2019) Toward understanding the origin and evolution of cellular organisms. Protein Sci. 28:1947-1951. doi.org/10.1002/pro.3715

24. Kanehisa M, Furumichi M, Sato Y, Ishiguro-Watanabe M, Tanabe M, (2021) KEGG: integrating viruses and cellular organisms. Nucleic Acids Res. 49:D545-D551. doi.org/10.1093/nar/gkaa970.

25. Overbeek R, Fonstein M, D'Souza M, Pusch G, Maltsev N, (1999) The use of gene clusters to infer functional coupling. Proc. Nat'l. Acad. Sci. 96:2896-2901. doi.org/10.1073/pnas.96.6.2896

26. The UniProt Consortium, (2019) UniProt: a worldwide hub of protein knowledge. Nucleic Acids Res. 47:D506-515. doi.org/10.1093/nar/gky 1049

27. Letunic I, Bork P, (2019) Interactive Tree of Life (iTOL) v4: recent updates and new developments. Nucleic Acids Res. 47:W256-W259. doi.org/10.1093/nar/gkz239

28. Huerta-Cepas J, Forslund K, Coelho LP, Szklarczyk D, Jensen L, Mering C, Bork P, (2017) Fast Genome-Wide Functional Annotation through Orthology Assignment by eggNOG-Mapper. Mol. Biol. Evol. 34:2115-2122. doi.org/10.1093/molbev/msx148

29. Huerta-Cepas J, et al. (2019) eggNOG 5.0: a hierarchical, functionally and phylogenetically annotated orthology resource based on 5090 organisms and 2502 viruses. Nucleic Acids Res. 47:D309-D314. doi.org/10.1093/nar/gky 1085

30. Klema V, Laub A, (1980) The singular value decomposition: Its computation and some applications. IEEE Transactions on Automatic Control 25:164-176. doi.org/10.1109/TAC.1980.1102314

31. NCBI Resource Coordinators (2018) Database resources of the National Center for Biotechnology Information. Nucleic Acids Res. 46:D8-D13. doi.org/10.1093/nar/gkv1290

32. The Gene Ontology Consortium (2020) The Gene Ontology resource: enriching a Gold mine. Nucleic Acids Res. 49:D325-334. doi.org/10.1093/nar/gkaa1113

33. Kesler I, et al. (2016) The EcoCyc database: reflecting new knowledge about Escherichia coli K-12. Nucleic Acids Res. 45:D543-550. doi.org/10.1093/nar/gkw1003

34. Cong Q, Anishchenko I, Ovchinnikov S, Baker D, (2019) Protein interaction networks revealed by proteome coevolution. Science 365:185-189. doi.org/10.1126/science.aaw6718

35. Schafer, J., and Strimmer, K. (2005). An empirical Bayes approach to inferring large-scale gene association networks. Bioinformatics 6, 185-189. doi.org/10.1093/bioinformatics/bti062

36. Sul, J.H., Martin, L.S., and Eskin, E. (2018). Population structure in genetic studies: confounding factors and mixed models. PLOS.
Genetics, http://doi.org/10.1371/journal.pgen. 1007309

37. Nagy, L.G., Merenyi, Z., Hegedus, B, Balint, B. (2020). Novel phylogenetic methods are needed for understanding gene function in the era of mega-scale genome sequencing. Nucl. Acids. Res. 48, 2209-2219. doi.org/10.1093/nar/gkz1241

38. Babu, M. et al. (2014). Quantitative genome-wide genetic interaction screens reveal global epistatic relationships of protein complexes in Escherichia coli. PLOS Genetics 10, e1004120. doi.org/10.1371/journal.pgen. 1004120

39. Babu, M. et al. (2018). Global landscape of cell envelope protein complexes in Escherichia coli. Nat. Biotechnol. 36, 103-112. doi.org/10.1038/nbt.4024

40. Hu, P. et al. (2009). Global functional atlas of Escherichia coli encompassing previously uncharacterized proteins. PLoS. Biol. 4, e100096. doi.org/10.1371/journal.pbio.1000096

41. Franceschini, A., Von Mering, C., Jensen, L.J. (2016). SVD-phy: improved prediction of protein functional associations through singular value decomposition of phylogenetic profiles. Bioinformatics 32, 1085-1087. doi.org/10.1093/bioinformatics/btv696

42. Mistry J, Finn R, (2007) Pfam: a domain-centric method for analyzing proteins and proteomes. Methods Mol. Biol. 396:43-58. doi.org/10.1007/978-1-59745-515-2_4

43. Huang, D.W., Sherma, B.T., Lempicki, R.A. (2009). Bioinformatics enrichment tools: paths towards the comprehensive functional analysis of large gene lists. Nucleic Acids Res. 37, 1-13. doi.org/10.1093/nar/gkn923

44. Huang, D.W., Sherma, B.T., Lempicki, R.A. (2009). Systematic and integrative analysis of large gene lists using DAVID Bioinformatics Resources. Nature Protoc. 4, 44-57. doi.org/10.1038/nprot.2008.211

45. Paul, K., Nieto, V., Carlquist, W.C., Blair, D.F., Harshey, R. (2010). The c-di-GMP binding protein YcgR controls flagellar motor direction and speed to affect chemotaxis by a “Backstop Brake” mechanism. Mol. Cell. 38, 128-139. doi.org/10.1016/j.molcel.2010.03.001

46. Walker, S.L., Redman, J.A., Elimelech, M. (2004). Role of cell surface lipopolysaccharides in Escherichia coli K12 adhesion and transport. Langmuir 18, 7736-7746 (2004). doi.org/10.1021/la049511f

47. Alm R, Mattick J (1995) Identification of a gene, pilV, required for type 4 fimbrial biogenesis in Pseudomonas aeruginosa, whose product possess a pre-pilin-like leader sequence. Mol. Microbiol. 16:485-496. doi.org/10.1111/j.1365-2958.1995.tb02413.x

48. Little A, et al. (2018) Pseudomonas aeruginosa AlgR phosphorylation status differentially regulates pyocyanin and pyoverdine production. mBio. 9:e02318-17. doi.org/10.1128/mBio.02318-17.

49. Kearns D, Robinson J, Shimkets L, (2001) Pseudomonas aeruginosa exhibits directed twitching motility up phosphatidylethanolamine gradients. J Bacteriol. 183:763-7. doi.org/10.1128/JB.183.2.763-767.2001

50. Rashid M, Kornberg A, (2000) Inorganic polyphosphate is needed for swimming, swarming, and twitching motilities of Pseudomonas aeruginosa. Proc. Natl. Acad. Sci. 97: 4885-4890. doi.org/10.1073/pnas.060030097

51. Wigner, E. P., (1967) Random matrices in physics. SIAM Rev., 9(1):1-23. doi.org/10.1137/1009001

52. Cover and Thomas (2006) Elements of information theory, 2nd edition. ISBN: 978-0-471-24195-9

1. A method of determining interaction between proteins, the method comprising:
receiving, at a first module, inputs corresponding to detectable proteomes of organisms, the inputs derived from sequencing genomes of the organisms;
generating, at the first module, a first matrix comprising a plurality of rows and a plurality of columns, where each of the plurality of rows corresponds to a detectable proteome of an organism, and where each of the plurality of columns corresponds to a genetic element, and where an entry in the first matrix represents a number of times each genetic element appears in each detectable proteome;
performing, at a second module, singular value decomposition (SVD) on the first matrix to generate a second matrix, where the second matrix is a spectral matrix representing a variation for each genetic element;
quantifying, at a third module, biological information contained in one or more cells of the spectral matrix by calculating pair-wise correlations for the genetic elements to obtain statistical interactions between the genetic elements at different scales of variation;
correlating, at a fourth module, the obtained statistical interactions with benchmarked biological interactions;
classifying, at a fifth module, a protein-protein interaction using the correlations between statistical interactions and the benchmarked biological interactions; and
displaying information indicating the classification to a user.

2. The method of claim 1, wherein the organisms comprise prokaryotic organisms or eukaryotic organisms.

3. The method of claim 2, wherein the organisms are prokaryotic organisms.

4. The method of claim 2, wherein the organisms are mammalian organisms.

5. The method of any of claims 1 to 4, wherein the genetic element includes an orthologous gene group.

6. The method of any of claims 1 to 5, wherein the genetic element includes a conserved protein domain.

7. The method of any of claims 1 to 6, wherein the SVD on the first matrix further produces a scaling for the spectral matrix showing that fractional variance of at least some components is linearly related to a component number, and wherein these components are used in the quantifying step.

8. The method of any of claims 1 to 7, wherein the pair-wise correlations for the genetic elements are calculated within all five-component windows of the one or more cells of the spectral matrix.

9. The method of any of claims 1 to 8, wherein the benchmarked biological interactions include phylogenetic relationships, indirect protein interactions in cellular pathways, direct protein interaction, or a mixture of indirect and direct interactions.

10. The method of any of claims 1 to 9, wherein the correlating step is conducted by quantifying mutual information shared between the statistical interactions and benchmarked biological interactions.

11. The method of any of claims 1 to 10, wherein the classifying is conducted using one or more trained Random Forest models.

12. The method of claim 11, wherein the one or more trained Random Forest models are trained via steps of:
dividing the spectral matrix obtained from the SVD into two or more mutual information windows, each of which is enriched for information representing at least one biological interaction; and
computing spectral correlations for each genetic element pair over each mutual information window.

13. The method of any of claims 11 and 12, wherein the one or more trained Random Forest models are trained using orthologous gene groups.

14. The method of any of claims 11 and 12, wherein the one or more trained Random Forest models are trained using conserved protein domains.

15. The method of any of claims 11 to 14, wherein the Random Forest models are trained using protein interaction information of E. coli K12.

16. A method of characterizing an emergent biological function, the method comprising:
developing, at a sixth module, a null model for protein correlations over a predetermined window of a spectral matrix using the method of claim 1;
identifying, at a seventh module, proteins that are statistically significantly correlated with a protein for the emergent biological function;
determining, at an eighth module, a spectral depth of correlation for all pairs of identified proteins by identifying a first window of the spectral matrix where correlation value for all pairs of the identified proteins is below the threshold of statistical significance;
identifying, at a ninth module, networks of proteins sharing a selected spectral depth of correlation to obtain statistical modules, wherein sets of proteins within each statistical module are connected to each other with a denser connection than to proteins outside the statistical module;
assigning, at a tenth module, a function to each of the statistical module using gene set enrichment analysis;
obtaining, at an eleventh module, hierarchical interactions in a pathway for the emergent biological function by comparing the assigned functions at various spectral depths; and
displaying information indicating the hierarchical interactions for the emergent biological function to a user.

17. The method of claim 16, wherein the predetermined window overlaps with an indirect interaction mutual information window for orthologous gene group variation.

18. The method of claim 17, wherein the predetermined window contains pathway-level interaction information.

19. The method of any of claims 16-18, further comprising, at a twelfth module, characterizing a protein in the statistical model as having the function of the statistical model based on the gene set enrichment analysis.

20. The method of claim 19, further comprising displaying information indicating the function of the protein to the user.

21. A non-transitory computer-readable storage medium storing instructions that, when executed by one or more processors, cause the one or more processors to perform operation for determining protein-protein interactions, the operations comprising:
receiving, at a first module, inputs corresponding to detectable proteomes of organisms, the inputs derived from sequencing genomes of the organisms;
generating, at the first module, a first matrix comprising a plurality of rows and a plurality of columns, where each of the plurality of rows corresponds to a detectable proteome of an organism, and where each of the plurality of columns corresponds to a genetic element, and where an entry in the first matrix represents a number of times each genetic element appears in each detectable proteome;
performing, at a second module, singular value decomposition (SVD) on the first matrix to generate a second matrix, where the second matrix is a spectral matrix representing a variation for each genetic element;
quantifying, at a third module, biological information contained in one or more cells of the spectral matrix by calculating pair-wise correlations for the genetic elements to obtain statistical interactions between the genetic elements at different scales of variation;
correlating, at a fourth module, the obtained statistical interactions with benchmarked biological interactions;
classifying, at a fifth module, a protein-protein interaction using the correlations between statistical interactions and the benchmarked biological interactions; and
displaying information indicating the classification to a user.

22. A non-transitory computer-readable storage medium storing instructions that, when executed by one or more processors, cause the one or more processors to perform operation for characterizing an emergent biological function, the operations comprising:
developing, at a sixth module a null model that is developed for protein correlations over a predetermined window of a spectral matrix using the method of claim 1;
identifying, at a seventh module, proteins that are statistically significantly correlated with a protein for the emergent biological function;
determining, at an eighth module, a spectral depth of correlation for all pairs of identified proteins by identifying a first window of the spectral matrix where correlation value for all pairs of the identified proteins is below the threshold of statistical significance;
identifying, at a ninth module, networks of proteins sharing a selected spectral depth of correlation to obtain statistical modules, wherein sets of proteins within each statistical module are connected to each other with a denser connection than to proteins outside the statistical module;
assigning, at a tenth module, a function to each of the statistical module using gene set enrichment analysis; and
obtaining, at an eleventh module, hierarchical interactions in a pathway for the emergent biological function by comparing the assigned functions at various spectral depths; and
displaying information indicating the hierarchical interactions in a pathway for the biological emergent function to a user.
